(19) United States
(12) Patent Application Publication
(10) Pub. No.: US 2004/0258614 A1
(43) Pub. Date: Dec. 23, 2004

(54) Microparticles for microarterial imaging and radiotherapy

(75) Inventors: Bruce R. Line, Cockeysville, MD (US); David A. Van Echo, Columbia, MD (US); Andrew S. Kennedy, Apex, NC (US); Hamidreza Ghandehari, Ellicott City, MD (US); Anjan Nan, Baltimore, MD (US)

Correspondence Address: SUGHRUE MION, PLLC, 2100 PENNSYLVANIA AVENUE, N.W., SUITE 800, WASHINGTON, DC, 20037, US

(73) Assignee: University of Maryland, Baltimore

(21) Appl. No.: 10/762,507

(22) Filed: Jan. 23, 2004

Related U.S. Application Data
(60) Provisional application No. 60/479,832, filed on Jun. 20, 2003.

(51) Int. Cl. A61K 51/00

(52) U.S. Cl. 424/1.11; 600/1

(57) ABSTRACT

Microparticles comprising a core, at least one linking carrier on the core, and at least one radioactive therapeutic agent covalently bonded to the linking carrier. The radioactive therapeutic agent may be a radionuclide or a radiopharmaceutical. A method of radiation therapy of a patient by administering to the patient the microparticles. The treatment may be radiation therapy to treat cancer or a tumor. A kit for preparing a microparticle treatment and a method for using the kit to prepare a microparticle treatment dose. The microparticle treatment dose may be made at a location of administration or at a site proximate to the location of administration, such as a local radiopharmacy, laboratory, hospital or physician's office.

### CROSS REFERENCE TO RELATED APPLICATION

This application claims benefit of priority of U.S. provisional application No. 60/479,832, entitled “Instant Microparticles for Microarterial Imaging and Radiotherapy”, filed on Jun. 20, 2003, which is incorporated by reference herein in its entirety.

### BACKGROUND OF THE INVENTION

Over 100,000 patients develop primary or metastatic cancer to the liver in the United States yearly. The majority of patients have surgically unresectable lesions that are poorly responsive to chemotherapy. Externally delivered conventional radiotherapy can cause hepatic tumors to regress or be destroyed, but selective delivery of radiation to predominately the tumor cells in the liver is almost impossible. Moreover, the dose of radiation required to destroy hepatic tumors far exceeds the tolerance of the normal liver cells immediately adjacent to the tumor. Thus a potentially important anti-cancer therapy, radiation, cannot now be used because of physical properties of photons (gamma radiation), and targeting limitations, leading to excessive radiation dose reduction for safety.

Therapy of Hepatic carcinoma

In the past decade significant resources have been expended in clinical trials testing hepatic artery infusion of chemotherapy, typically FUDR or 5FU. [1, 32, 33] Despite eight prospective randomized trials in patients with colorectal cancer liver metastases, there is yet no consensus as to the efficacy and optimal use of this approach. With other diseases such as carcinoid, hepatoma, breast, lung and sarcoma, non-surgical management of liver lesions is the mainstay, producing palliation at best with short prolongation of survival. It has long been understood that chemosensitization for many solid tumors is beneficial. However, it is unclear how to optimally deliver hepatic radiation while respecting normal tissue tolerance. Brachytherapy offers the hope of delivering tumorcidal doses. Surgical resection is obviated with diffuse hepatic involvement or where extra-hepatic failure is likely. Interesting approaches with radioactive seed placement which predate advancements in 3D external beam treatment planning are published, but are not currently in practice.[34] Radiofrequency ablation, among other local therapies, is less invasive, but effective in these special cases of limited, focal tumors that would be amenable to a seed implant approach. [35-38]

Despite recent advances with chemotherapy, surgery and interventional radiology in treating solid tumors in the liver, it remains a significant site of refractory disease in solid tumors. Conservative estimates of liver disease in colorectal cancers alone represent at least 77,500 cases in the USA yearly. [1] With the addition of pancreas, carcinoid, stomach and other solid tumors, the total number of patients with malignant liver tumors is in excess of 150,000. [2] In addition, the incidence of hepatocellular cancer in the United States and elsewhere is increasing, with a US rate of 2.4 per 100,000 between 1991 and 1995. [3] Most of these patients will not have curative surgical therapy and need new therapy options, with radiation therapy providing potential benefit.4 Delivering sufficiently high doses of radiation via external beam to destroy metastatic or primary liver tumors is usually not possible due to the relatively low radiation tolerance of hepatocytes. In seminal work by Lawrence, it was demonstrated that, with conformal three-dimensional treatment with concurrent hepatic artery chemotherapy, radiation given in the same dose ranges as is delivered to non-hepatic sites can produce durable hepatic tumor control, without loss of hepatic function [4-8] However, most patients are not candidates for this or other localized therapy such as radiofrequency ablation, cryotherapy or chemoembolization, due to many factors. Therefore, a technique is needed to deliver high doses of radiation to tumors in the highly sensitive liver, which will be broadly applicable due to its ability to spare normal liver from serious injury.

Brachytherapy, placing the radiation directly in the tumor, is an attractive option. Ideally, a non-surgical approach, given as in outpatient, could provide therapy to a large number of patients safely while using available interventional radiology techniques and catheters. A beta-emitting isotope would allow for a sharp dose fall off in tissue to enable sparing of adjacent normal cells. But this is also a significant drawback, as the short-range electrons cannot be imaged or confirmed in anyway as to their location.

Embolization is a process wherein a material is injected into a blood vessel to at least partially fill or plug the blood vessel and/or encourage clot formation so that blood flow through the vessel is reduced or stopped. Embolization of a blood vessel can be useful for a variety of medical reasons, including preventing or controlling bleeding due to lesions (e.g., organ bleeding, gastrointestinal bleeding, vascular bleeding, and bleeding associated with an aneurysm), or to ablate diseased tissue (e.g., tumors, vascular malformations, hemorragic processes, etc.), by cutting off blood supply. Embolization may also be used to prevent blood loss during or immediately following surgery. Embolization of tumors may be performed preoperatively to shrink tumor size, to aid in visualization of a tumor, and to prevent blood loss related to surgical procedures.

Pioneering efforts in vascular embolic agents with and without radiation can be traced back to Prinzmetal, [39] who first showed the utility of glass sphere infusion via arterial routes in animal studies and human subjects in 1947. Shortly after, Muller in 1951 used intravenously injected radioactive gold in charcoal to treata patient with bilateral lung cancer. Moreover, multiple researchers in the early 1960's, who were treating highly vascular neuroendocrine tumors in the liver, discovered efficacy with hepatic artery infusion of 90Yttrium-resin spheres of approximately 35 μm. Unfortunately, they also discovered that fatal toxicities related to unintended deposition of spheres in the stomach causing ulceration and hemorrhage could occur despite careful planning and technique. Subsequent researchers also described fatal pulmonary toxicity from radiation pneumonitis from shunting of radioactive spheres from the liver to the lungs, the next capillary bed after the liver. Fortunately, from modern research in Canada using glass microspheres, in Australia using resin spheres, and from our experience, the incidence of GI bleed, biliary sclerosis, or pulmonary toxicity is extremely low.[21, 22, 24-26, 28, 29, 31, 40-44]

The use of beta radiation in the liver is not a new concept, with attempts to deliver 90Y or 32P attached to resin or ceramic microspheres in the early 1960's. [9-15] All groups eventually switched to 90Y for its relatively high beta energy compared to other isotopes. Yttrium-90 (⁹⁰Y) is a pure beta emitter, which decays to stable zirconium-90 with an average energy of 0.94 MeV via a half-life of 2.67 days (64.2 hours). It is produced by neutron bombardment of ⁸⁹Y in a commercial reactor, yielding ⁹⁰Y beta radiation with a tissue penetration of 2.5 mm, and a maximum range of 1.1 cm. One GBq (27 mCi) of ⁹⁰Y delivers a total dose of 50 Gy/Kg in tissue.

Radioactive Microparticles Background

Many previous attempts have been made to locally administer radioactive materials to patients with cancer as a form of therapy. In some of these, the radioactive materials have been incorporated into small particles, seeds, wires and similar related configurations that can be directly implanted into the cancer. In other approaches, the radioactive materials have been formulated into microspheres of regular size for injection into the arterial blood supply of the target organ. When radioactive particles or microspheres are administered into the blood supply of the target organ, the technique has become known as Selective Internal Radiation Therapy (SIRT). Generally, the main application of SIRT has been to treat cancers in the liver.

In the earliest clinical use of yttrium-90-containing microspheres, the yttrium was incorporated into a polymeric matrix that was formulated into microspheres. While these microspheres were of an appropriate density to ensure good distribution characteristics in the liver, there were several instances in which the yttrium-90 leached from the microspheres and caused inappropriate radiation of other tissues.

In one attempt to overcome the problem of leaching, a radioactive microsphere comprising a biologically compatible glass material containing a beta- or gamma-radiation emitting radioisotope such as yttrium-90 distributed throughout the glass, has been developed (International Patent Publication No. WO 86/03124).

There have been several reports of clinical studies on the use of solid glass radioactive microspheres. In one report, ten patients with primary hepatocellular carcinoma were treated, however no patient had a complete or partial response (Shepherd, F. et al., Cancer, Nov. 1, 1992, Vol.70, No.9, pp 2250-2254).

A further development in order to overcome the problem of leaching, was the production of light polymeric ion-exchange microspheres that did not leach their yttrium content when injected into the body. Using these microspheres, a high objective response rate for patients with secondary cancer in the liver was obtained when the microspheres were injected into the hepatic artery (Gray, B. N. et al. Regression of liver metastases following treatment with Yttrium-90 microspheres. Aust. N. Z. J. Surg. 1992, 62:105-110). One disadvantage of such polymeric ion exchange microspheres is that the yttrium-90 radionuclide must be added to the microsphere after neutron activation of the stable isotope of yttrium-89. This requires the use of specialised facilities and potentially is hazardous to the manufacturing personnel. Furthermore, the polymeric microspheres contain only a low percentage of yttrium.

Using the technique described by Gray et al., other clinical studies in patients with secondary liver cancer have demonstrated a very high response rate using yttrium-90 containing microspheres. In one study in patients with metastatic liver cancer, the majority of patients benefited from treatment with radioactive microspheres with appropriate physical characteristics, especially when combined with perfusion of cytotoxic drugs into the arterial circulation of the liver (Gray, B. N. et al., supra).

There have been modifications of the ⁹⁰Y carrier which include resin-based and ceramic types. Hafeli and Day reported a modification of a glass microsphere (magnesium alumino borate glass, 25-32 μm) to replace ⁹⁰Y with Rhenium (natural isotopes ¹⁸⁶Re and ¹⁸⁷Re). These have large cross-sections for neutrons, and more easily yield therapeutic amounts of beta-emitters ¹⁸⁶Re and ¹⁸⁸Re, which have maximal energies of 1.1 MeV and 2.1 MeV respectively, compared with ⁹⁰Y of 0.97 MeV. The purpose of using them, however, was that the γ-rays released were 9.5% (¹⁸⁶Fe) and 15%(¹⁸⁸Re). They reported its use in Sprague-Dawley rats with Novikoff hepatoma, a highly chemo- and radioresistant tumor, but no human studies have been published. Interestingly, the γ-ray production made imaging possible, perhaps for human use. However, the very short half-life of ¹⁸⁶⁺¹⁸⁸Re (17 hours) compared to ⁹⁰Y (65 hours) makes ¹⁸⁶⁺¹⁸⁸Re questionable for clinical practice. Other isotopes have been labeled to a variety of spheres; none are currently in human trials with the exception of ³²P used exclusively in China in the early 1990's, although there are no current publications.

Patient Selection and Evaluation

Patients who are generally appropriate to treat using the microsphere therapies include patients who had already received, and failed, standard first and second line therapies. Specialists from Medical Oncology, Radiation Oncology, and Interventional Radiology evaluate each patient prior to acceptance into the therapeutic protocol. Patients are usually selected if they are candidates for treatment, and are ≧18 years of age, and have a confirmed diagnosis of a non-hematologic malignancy, with measurable unresectable disease, predominately involving the liver. Patients are able to give informed consent, with ECOG Performance Status score of less than or equal to 2. They have adequate bone marrow (granulocytes >1500/μl, platelets >60,000/μl) hepatic (bilirubin ≦2.0 mg/dl, SGOT/SGPT or Alkaline Phosphatase <5 times the upper limit of normal), and pulmonary function (FEV₁ >1L). There must not be contraindications for angiography and selective visceral catheterization or pulmonary shunt of >10% or any flow to the GI tract. Pregnant women are excluded, so are patients with hepatofugal blood flow, complete portal vein thrombosis or previous pulmonary irradiation from any source with an estimated absorbed dose >30 Gy. Patients needing systemic chemotherapy within 4 weeks of treatment are also not considered appropriate for this type of treatment.

A complete history and physical was performed by the treatment team (Radiation Oncologist, Medical Oncologist, and Interventional Radiologist co-investigators) prior to making a final decision as to microsphere therapy. Appropriate blood work includes liver function tests, electrolytes, complete blood count with differential, PT, PTT, INR, lipase, and appropriate tumor markers for their malignancy (CEA, AFP, CA 19-9, Chromogranin A, CA 27-29, 5-HIAA, etc.). All of these laboratory parameters are repeated weekly post-infusion for 8 weeks, then monthly thereafter to monitor for toxicity.

All patients are evaluated via chest, abdomen and pelvic CT scans regardless of tumor type, in an effort to detect extra hepatic metastases. Hepatocellular carcinoma patients typically also undergo MRI with contrast of the liver to better define tumor location, size and number. To further assist tumor burden and response to therapy, all patient with neuroendocrine tumors undergo In⁻¹¹¹ Octreoscan (OctreoScanTM Kit, Indium In⁻¹¹¹ Pentetreotide, Mallinckrodt Medical Inc., St. Louis, Mo., USA). All other non-hepatoma patients undergo FDG-PET scanning as a pretreatment and post-treatment routine examination. A non-contrast CT scan of the liver was performed in the Radiation Oncology department for dosimetry pre-planning based on 3-dimensional reconstruction of the liver. Volumetric data was then used in calculating the correct activity of microspheres for an individual patient. Dose distribution calculations based on the microsphere distributions estimated with MAA-SPECT scans are aided via registration to these planning CT scans.

All patients undergo mapping of the celiac, aortic and hepatic vasculature via femoral catheter approach. The treatment team reviewed the most appropriate delivery routes and determined hepatic volumes supplied by the right or left hepatic arteries. This was essential in aiding pre-treatment planning and dosimetry calculations. Typically, the angiogram is performed the week before treatment, but on occasion, it is done up to 3 weeks prior to the actual delivery of microsphere therapy. If it were determined during the angiogram that the gastroduodenal artery would pose a significant opportunity for escape of microspheres into the GI tract, coil embolization would be performed. In two cases, the tumor had parasitized arteries near the diaphragm, which were embolized to minimize deposition of microspheres along the diaphragm.

Shunt Evaluation

All patients are tested for an occult shunt from the hepatic arterial system to the pulmonary or gastrointestinal venous systems via planar and SPECT imaging of 4.5-6.0-mCi ^{99m}Tc-labeled macro aggregated albumin (MAA). The MAA particles approximate the size of the microspheres, but can be imaged and quantified easily via a gamma camera. Each ^{99m}Tc-MAA infusion typically contains 3.6-6.5 million particles, with >85% between 20 μm and 40 μm (Package insert of Pulmolite®-CIS-US, Inc, 10 DeAngelo Drive, Bedford, Mass., USA). Planar and SPECT imaging was performed on all patients to better determine if a shunt was present. The protocol outlined an upper limit for cumulative total dose to the lungs of 30 Gy or 16.5 mCi. The patient would be disqualified from the study to prevent pulmonary toxicity if an absolute shunt value of 10% of the infused Tc99m MAA activity on any screening study was detected in the lungs or if anatomic shunting was detected in the GI tract. Because the shunt fraction estimate is significantly affected by the estimation procedure used, we chose a geometric mean analysis with a liberal hepatic region of interest (ROI). The liberal hepatic ROI was obtained by increasing the image intensity to include most of the scatter originating from that organ. All ROI counts were corrected for background obtained from the abdominal region well below the liver and avoiding the urinary tract. Regions of interest were drawn around the liver and lungs in both anterior and posterior whole body planar images, and the shunt was calculated using:

$$\text{Shunt Fraction} = \frac{ROI \text{Lung counts}}{ROI \text{Lung counts} + ROI \text{Liver counts}} .$$

SPECT imaging was performed to better determine if a gastrointestinal shunt was present and to provide three-dimensional data to correlate with pre and post therapy PET scans.

Within 24 hours of microsphere infusion, all patients returned to the Nuclear Medicine department for acquisition of planar and torso SPECT images produced from the microspheres themselves by release of Bremsstrahlung (gamma) radiation. This quality assurance test confirmed that the radiation dose was deposited only in the liver, and was compared to the distribution of activity found on the pretreatment ^{99m}Tc-MAA scans.

Radiation Treatment Planning

In a typical patient evaluation, patients undergo CT treatment planning in the Radiation Oncology department with reconstruction of the liver volumes (whole liver, right lobe, and left lobe) from the liver contours delineated by the Radiation Oncologist, using the AcQ-sim v.4.0 software (Picker International, Inc., 595 Miner Rd. Highland Hts., Ohio 44143). CT scans are performed without IV or oral contrast, using 3 mm slice thickness and breath hold by the patient during liver imaging. The required activity to be ordered for each patient is calculated based on a nominal target dose of 150 Gy and patient's liver mass determined from the AcQ-sim data, assuming the uniform distribution of the microsphere throughout liver volume as (Package Insert, TheraSphere®, MDS Nordion, Inc., 447 March Road, Ontario, Canada K2K 1X8):

$$A ( GBq ) = \frac{D ( Gy ) \times M ( Kg )}{50}$$

where A is the activity, D is the nominal target dose, and M is the liver mass. For a typical patient with liver mass of 2 Kg, the required activity is 6 GBq. Keeping the lung dose below 30 Gy to prevent radiation pneumonitis restricted patient selection to those with ≦10% shunt fraction. With the use of glass or ceramic microspheres, the microspheres must be ordered from the supplier, and the patient is scheduled for treatment allowing for the appropriate decay from the calibration time. Such microspheres are delivered in a sealed vial encased in 1.2 cm thick plexiglass cylinder.

The activity of the microspheres is verified upon receiving the delivery from the manufacturer by measuring the Bremsstrahlung radiation from outside of the plexiglass cylinder at a fixed distance of 30 cm using a radiation survey meter. The measurement serves as a consistency check as well as the baseline value before the infusion procedure. After infusion, the microsphere vial and the delivery catheters and lines are put in a large plexiglass container of the same thickness and measured at the same distance. The values measured before and after infusion provide the percentage of microspheres and the nominal dose being delivered to the patient.

A key safety feature of the screening procedures, particularly the MAA scan, is to prevent high doses of radiation to the GI tract or lungs. Because shunts are not readily seen on angiogram, CT or MRI, MAA scans are used to assess the extent of shunt. However, the underlying principle regarding MAA is that because of its size, it will simulate the deposition of glass microspheres. This has been questioned by us and others [40] because the specific gravity of the glass spheres is significantly more than saline or the MAA particles. While it seems this may be of concern, no patient in prior clinical trials using these glass spheres has experienced a significant pulmonary toxicity when the MAA showed a shunt fraction less than 15%. Moreover, the purpose of the MAA—to screen for shunts—is reliably accomplished, but neither the shape nor the weight of the albumin particles closely resembles the glass microspheres.

Dosimetry can currently not be performed with infusions of this type; rather, the total activity of the spheres infused is recorded. Limited attempts [20, 45-47] in the past to develop dosimetry models have included pathology samples and nuclear medicine images, but not modem radiation therapy algorithms. We have recently presented the first dose-volume histograms and 3D isodose volumes for microsphere therapies.[30] However, they are based on MAA data that may not be closely related to the actual glass microsphere deposition.

Microsphere Administration

Microsphere therapy administration is performed in an outpatient angiography suite with a radiation physicist present to survey staff members exiting the treatment suite and to monitor any possible contamination. The floor of the angiography room is covered with large drapes before the treatment to confine any potential contamination. All contaminated materials (e.g. drapes, gloves, shoe covers, etc) are collected and disposed of as radiation waste.

The interventional radiologist places a catheter percutaneous via patient's femoral artery into the proper hepatic artery. The physicist, radiation oncologist and interventional radiologist independently confirm the proper identification of the patient, dose to be delivered, lobe or whole liver to be infused, and activity of the microspheres to be infused. The radiation oncologist performing the infusion reviews the catheter position with the interventional radiologist. The infusion flow rate of microspheres (1-2 cc/second typically), is set to avoid reflux of spheres back into the gastric artery supply. The microsphere vial is connected to an administration device routes the microspheres from the vial into the patient's catheter. The radiation oncologist performs each infusion in successive flushes of 10 to 20 cc. A radiation exposure meter is placed next to the source vial to assess the remaining activity within the vial. The physicist monitors the exposure through the entire system during infusion using a directional GM counter. He also informs the radiation oncologist when the maximum activity transfer has been achieved. An infusion of higher than 95% of the total dose is desired. The used vial and connecting catheters are disposed of in a plexiglas jar and then measured for residual activity. The actual activity delivered to the patient is determined from the ratio of exposure from the plexiglas jar to the exposure from the microsphere vial upon receiving the source (normalized to the same distance and corrected for the decay).

The microsphere infusion set has allowed safe delivery of microspheres. There are potential pitfalls, however, due to the complexity of the system, that can cause miss-timing between the saline infusion into the microsphere chamber, the purging of air in the line, the waste vial, and the microsphere infusion into the patient. If a portion of the dose flows into the waste vial, there is no way to recover it. If this occurs; the patient may need to receive an additional infusion of microspheres. The company offering this therapy product is developing a replacement infusion set that will address this issue.

All patients recover from anesthesia in a private room in interventional radiology. Measurements of exposure rates at a distance from the patient's liver are obtained to determine the number of days the patient would need to avoid contact (<3 feet) with others. For nearly all patients, this was 3 days after infusion for adults, and 14 days regarding children and pregnant women. Most patients were able to have the femoral artery site closed by suture, and therefore could be discharged 2 hours after completion of the infusion.

### BRIEF SUMMARY OF THE INVENTION

The present invention relates to an agent introduced by intravascular administration for diagnostic and/or therapeutic intervention. The agent consists of or contains a suspension of microscopic radioactive particles in a physiologically acceptable liquid for injection into humans.

To achieve safe and effective treatment, Applicants have developed methods to irradiate hepatic cancers internally with 90-Yttrium radiotherapy (beta radiation). It overcomes the three major limitations of external radiotherapy: 1) utilizing beta radiation which will only penetrate a few millimeters from the sphere containing it, thus sparing normal nearby hepatocytes, and 2) delivery is directly to the tumor vasculature where it will reside within the tumor, also sparing normal liver near the tumor, enabling 3) significantly increased radiation dose delivery is possible, to achieve the range of radiation known to be effective in destroying virtually all solid tumors. Although this therapy has provided significant benefit to over 100 patients at the University of Maryland, and yielded one of the largest single-institutional experiences with this type of therapy.

Beta irradiation from 90-Yttrium internal sources results in exposure to both tumor tissue and normal liver because the range of the irradiation is over 1 cm. Alpha irradiation is an attractive potential therapeutic isotope in conjunction with, or in place of 90-Yttrium, given alpha radiation's much shorter-range (microns) in tissue, and much higher radiation activity over this very short distance. The present invention also includes coupling alpha emitter radionuclides to microsphere particles, alone in conjunction with gamma and beta emitting isotopes or with alpha particles. The particles may be infused via the hepatic artery and become trapped in the liver tumor capillary bed. This approach will allow the delivery of high intensity tumoricidal doses of radiation without exposure of normal liver tissue. Such a therapy has the potential to cure hepatic tumors that are otherwise uniformly fatal.

There are many potential advantages of SIRT over conventional, external beam radiotherapy. Firstly, the radiation is delivered preferentially to the cancer within the target organ. Secondly, the radiation is slowly and continually delivered as the radionuclide decays. Thirdly, by manipulating the arterial blood supply with vasoactive substances (such as Angiotensin-2), it is possible to enhance the percentage of radioactive microspheres that go to the cancerous part of the organ, as opposed to the healthy normal tissues. This has the effect of preferentially increasing the radiation dose to the cancer while maintaining the radiation dose to the normal tissues at a lower level (Burton, M. A. et al.; Effect of Angiotensin-2 on blood flow in the transplanted sheep squamous cell carcinoma. Europ. J. Cancer Clin. Oncol. 1988, 24(8):1373-1376).

When microspheres or other small particles are administered into the arterial blood supply of a target organ, it is desirable to have them of a size, shape and density that results in the optimal homogeneous distribution within the target organ. If the microspheres or small particles do not distribute evenly, and as a function of the absolute arterial blood flow, then they may accumulate in excessive numbers in some areas and cause focal areas of excessive radiation. It has been shown that microspheres of approximately 25-50 micron in diameter have the best distribution characteristics when administered into the arterial circulation of the liver (Meade, V. et al; Distribution of different sized microspheres in experimental hepatic tumours. Europ. J. Cancer & Clin. Oncol. 1987, 23:23-41).

If the microspheres or small particles do not contain sufficient ionising radiation, then an excessive number will be required to deliver the required radiation dose to the target organ. It has been shown that if large numbers of microspheres are administered into the arterial supply of the liver, then they accumulate in and block the small arteries leading to the tumour, rather than distribute evenly in the capillaries and precapillary arterioles of the tumour. Therefore, it is desirable to use the minimum number of microspheres that will provide an even distribution in the vascular network of the tumour circulation.

Similarly if the microspheres or small particles are too dense or heavy, then they will not distribute evenly in the target organ and will accumulate in excessive concentrations in parts of the liver that do not contain the cancer. It has been shown that solid heavy microspheres distribute poorly within the parenchyma of the liver when injected into the arterial supply of the liver. This, in turn, decreases the effective radiation reaching the cancer in the target organ, which decreases the ability of the radioactive microspheres to kill the tumour cells. In contrast, microspheres distribute well within the liver (Burton, M. A. et al.; Selective International Radiation Therapy; Distribution of radiation in the liver. Europ. J. Cancer Clin. Oncol. 1989, 25:1487-1491).

For radioactive microspheres to be used successfully for the treatment of cancer, the radiation emitted from the microspheres should be of high energy and short range. This ensures that the energy emitted from the microspheres will be deposited into the tissues immediately around the microspheres and not into tissues which are not the target of the radiation treatment. There are many radionuclides that can be incorporated into microspheres that can be used for SIRT. Of particular suitability for use in this form of treatment are the unstable isotopes of yttrium (Y-90) and phosphorous (P-32), although other isotopes such as iodine can also be used. Yttrium-90 is the unstable isotope of yttrium-89 which can be manufactured by placing the stable yttrium-89 in a neutron beam. The yttrium-90 that is generated decays with a half life of 64 hours, while emitting a high energy pure beta radiation.

If the microspheres contain other radioactive substances that are not required for the radiation treatment of the target tissue, then unwanted and deleterious radiation effects may occur. It is therefore desirable to have microspheres of such a composition that they only contain the single desired radionuclide. In this treatment mode, it is desirable to have microspheres that emit high energy but short penetration beta-radiation which will confine the radiation effects to the immediate vicinity of the microspheres. For this purpose, yttrium-90 is the preferred radionuclide.

Therefore, the ideal microspheres for use in this treatment mode will have a low density relative to pure yttria, be in the size range of from 20-80 micron, and be stable so that no material leaches from the microspheres when administered into the body of a human or other mammalian patient.

Current Therapy with Y90 Microspheres

Early efforts to use microspheres to deliver radiation into the liver focused on hepatocellular, carcinoid and colorectal tumor types. [9-26] Much has been learned regarding protecting patients from sometimes fatal pulmonary deposition of microspheres via abnormal shunting to the next vascular bed with mean vessel diameters less than 25 μm. [27, 28] There was no pursuit of this therapeutic approach in the United States after the 1960's until investigations using a glass microsphere (TheraSphere®, MDS Nordion, Kanata, Ottawa, Canada) developed at the University of Missouri, and brought to clinical practice by Andrews et al at the University of Michigan in 1994.25 Unfortunately, after only a brief attempt to study toxicity in a phase I trial, use of microsphere therapy again disappeared from the USA until its reintroduction by the GI Oncology Team at the University of Maryland. 29-40 41-43 Resin-based ⁹⁰Y microspheres (SIRTeX® Medical, Centrecourt Business Park, Unit D4, 25-27 Paul St. North, North Ryde, NSW, Australia 2113) once available exclusively in Asia, received FDA approval (PMA) for commercial use in the USA for colorectal cancer in February 2002. However, at the time we developed our liver-directed radiotherapy program, we pursued the only product that was available in North America, which was composed of glass, previously used by Andrews et al. 25.

In designing a new program to deliver microspheres, we desired to avoid any surgical procedures, ports, hepatic artery pumps, or scheduled admissions such has been done previously in most studies. Our goal was to have patients go home ambulatory the same day as the treatment with few side effects; especially no toxicities such as are common post-embolization. We also wanted to improve upon some of the basic infusion techniques, pre-planning, and dose determination procedures, and take advantage of modem imaging modalities, such as Tc99m MAA-SPECT, PET and CT/MRI. We also wanted to ensure that all patients were evaluated by a multidisciplinary team of GI cancer specialists to ensure those patients selected for microsphere therapy were most appropriate, which was not always the situation with prior reports of microsphere therapy results.

### BRIEF DESCRIPTION OF THE DRAWINGS

FIG. 1: A typical example of microsphere construction from a biocompatible core chemically linked to a chelator that tightly binds a radioactive emitter. The radioactive isotope can be supported by a dendrimeric interface to increase the amount of bound chelator.

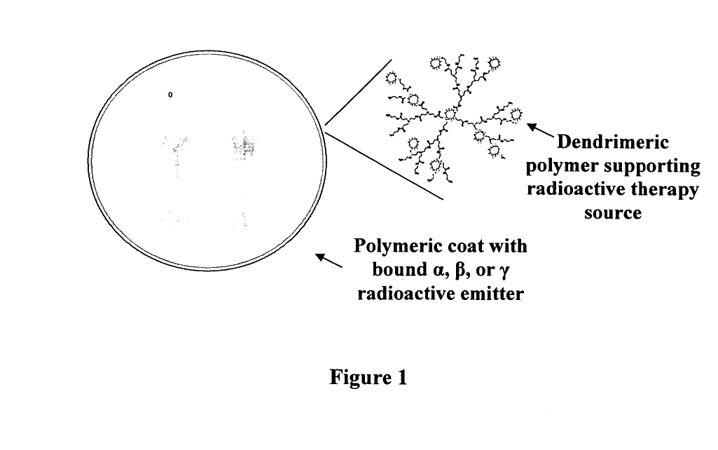

FIG. 2. Synthesis of PMMA-PAMAM dendrimer conjugate.

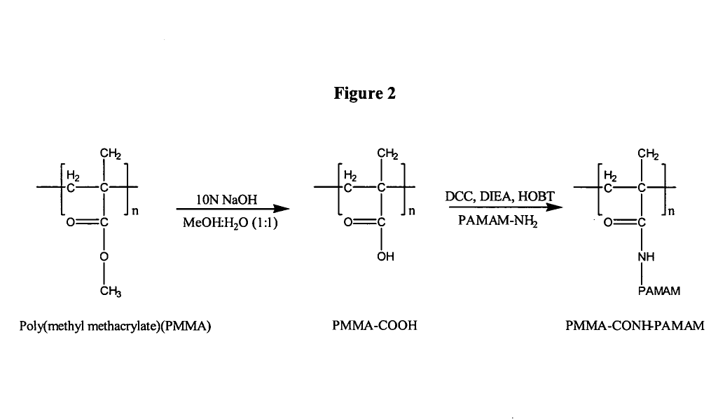

FIG. 3. Synthesis of PMMA-DOTA microspheres.

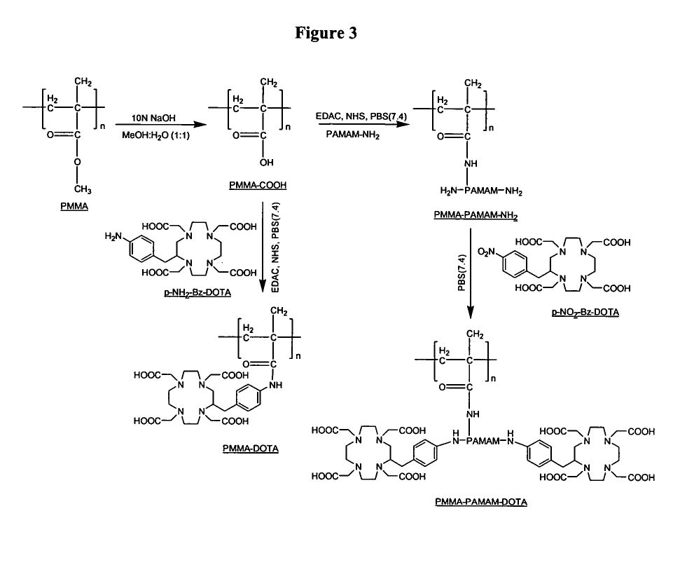

FIG. 4. Microparticle.

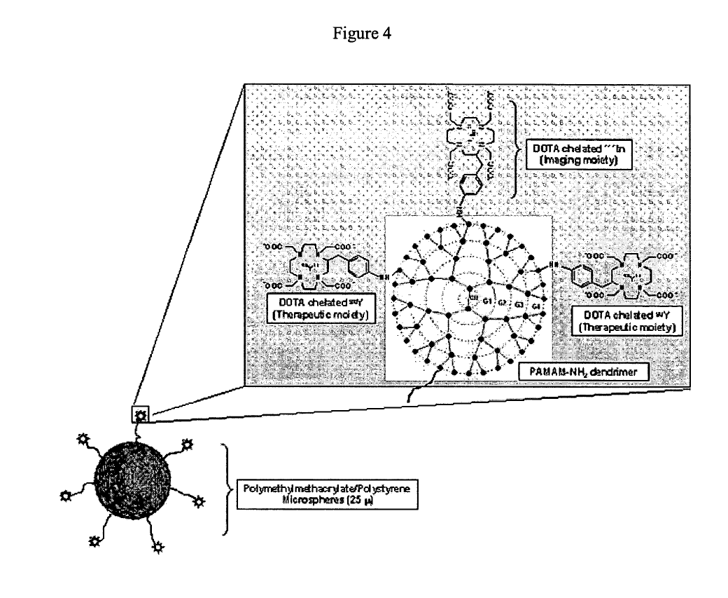

### DETAILED DESCRIPTION OF THE INVENTION

The present invention relates to the radioactive complex formed by labeling a biocompatible polymer, with radionuclide, a microsphere/particle core that supports the polymer-radioactive complex forming micro particles, and a “kit” of the necessary components for preparing the radioactive complex. In addition, the present invention relates the process of preparation thereof and methods and use thereof for an internal radiation diagnostic and/or therapeutic agent.

A particulate material is used (polymethacrylate requiring chemical alteration with DCC or DMAP before it can be used), which comprises biocompatible microspheres having a diameter in the range of from 5 to 200 microns. The material may have attached an alpha, beta- or gamma-emitting radionuclide, or all three, depending upon the clinical need.

The microsphere is comprised of a non-ceramic, non-radioactively labeled core material that serves as a support for a polymeric coating comprised of a linear, branched, or dendromer biocompatible polymer to which a suitable binding agent is attached. The binding agent is selected from a number of chemically stable compounds that bind radioactive or non-radioactive therapeutic agents.

In the present invention, locally deposited polymer depots on the surface of the microsphere core are used as a vehicle for the immobilization and local delivery of a radionuclide or radiopharmaceutical.

Standard radionuclides that have been used for local radiotherapy (brachytherapy) may be used, such as radionuclides of rhenium, iodine, iridium, radium, cesium, yttrium or other elements.

Suitable therapeutic and diagnostic agents include those whose efficacy within the body is predicated on their ability to remain within or to be carried within the vascular compartment. Accordingly this method can be readily adapted for treating several diseases and disorders, including cancer and for imaging selected regions of a mammal (humans) by various imaging techniques.

The chemical durability of the microspheres is such that they do not release a significant amount of radiation emitting radioisotope into the circulatory system upon administration.

Microspheres or particles may contain, via chelate attachment to the linear, branched or dendrimeric polymer coat, any or all of the following: phosphorus, yttrium, rhenium, and other beta emitting isotopes; actinium, bismuth, astatine and other alpha emitting isotopes; technetium, indium, iodine and other gamma emitting isotopes; and carbon, nitrogen, fluorine, sodium, magnesium, aluminum, silicon, potassium, vanadium, manganese, gallium, niobium, iodine and/or lead.

To deliver a controlled dosage of an insoluble material into a living body, the material is provided in measured amounts in vials, and a system is provided for flushing the entire content of insoluble materials incorporating radioactive isotopes from the vial into the body.

An accurate dosage of radioactivity can be delivered by administering the entire contents of one vial according to the radioactivity in selected vial as determined by the initial measurement of radioactivity and by the natural half-life of the isotope.

The radioactive complex can be used as an internal radiation therapy for hepatic cancer, rheumatoid arthritis or solid cancers such as liver cancer, brain cancer, breast cancer, ovary cancer and the like by administering them directly to the lesion through vascular routes.

The current invention embodies a material and procedure to internally irradiate hepatic cancers. The invention overcomes the three major limitations of external radiotherapy:

1) It utilizes radiation which will only penetrate a short distance from the sphere containing it, thus sparing normal nearby hepatocytes;

2) It is delivered directly to the tumor vasculature where it will reside within the tumor, also sparing normal liver near the tumor; and

3) It enables significantly increased radiation dose delivery, to achieve the range of radiation known to be effective in destroying virtually all solid tumors.

Local radiotherapy can be applied to any tumor which is accessible by a vascular catheter. This technique is particularly applicable to either highly vascularized tumors or tumors which have a single dominant arterial vascular supply. This would provide a method for treatment particularly applicable to renal cell carcinoma, hepatoma, sarcomas, cancers of the head and neck, and central nervous system tumors. For example, radioactive microspheres containing yttrium-90 are may be injected into the artery supplying a tumor. The local tumor volume in the area of deposition is radiated while the microspheres are immobilized at the site of deposition.

The microspheres can be chosen for a longer time of degradation or elimination of greater than 320 hours, when five half-lives of the implanted yttrium-90 have expired and the vast majority of radioactive decay has occurred.

Radioactive microspheres that have been used clinically (TheraSphere®) (MDS Nordion, Inc., 447 March Road, Ontario, Canada K2K 1X8) are composed of glass impregnated with ⁹⁰Y. Each sphere has a diameter of 25±10 μm so they are trapped mainly within tumor terminal arterioles, which are estimated to have a diameter of 8-10 μm. It is estimated that each milligram contains between 22,000 and 73,000 microspheres. The ⁹⁰Y does not leach from the glass spheres in the patient, because it is permanently trapped in the matrix of the microsphere.

There are significant deficiencies in the two products currently available for microsphere therapy. First, there is no gamma source on the spheres for imaging to ensure their location in the patient is confirmed. This also complicates attempts to develop radiation treatment planning software. Clinical trials and broad use of this type of device will require accurate dosimetry and localizing within the liver, as is current state of the art for any brachytherapy product. Second, the process of generating ⁹⁰Y spheres is cumbersome, requiring shipments from nuclear reactors with some capacity in South Africa, Missouri, and Australia, which with some frequency in our experience do not arrive at the hospital at the optimal time to be used based on isotope decay. It has been stated by production staff in both manufacturers (Personal communication with A. Kennedy with MDS Nordion and SirTex), that they can only produce and deliver a maximum of 4,000 doses combined. There is a need for a more efficient production and delivery system, which would allow more patients to receive therapy in a timely manner. Because there is fixed activity at the time of manufacture, there is only a 4-hour window for use of glass spheres, and <24 hours for resin spheres.

The invention described in detail below takes advantage of the expertise and equipment available in hospitals with nuclear medicine. The invention is comprised of a “kit” of polymer spheres, linkers, and isotope, that is mixed onsite or in a local radiopharmacy. The advantage entails increased flexibility, with a dose tailored to individual patient needs based on preplanning dosimety. The invention provides for single or multiple tracer tags with gamma and beta or alpha sources. The hallmark of this approach is its answer to the two most problematic issues with existing microspheres, i.e. lack of imaging of spheres, and limited distribution of product.

The present invention provides microparticle constructs comprising a biocompatible microparticulate core, an optional linking carrier, and an molecular effector coupled directly or indirectly to the biocompatible core. A preferred form of the effector as a radioisotope bound to the linking carrier by a chelator group. In addition, the present invention includes the process of preparation of a “kit” formulation thereof and the use thereof for an internal radiation diagnostic and/or therapeutic agent.

A particulate material is used, which comprises biocompatible microspheres having a diameter in the range of from 10 to 200 microns.

The microsphere is comprised of a non-ceramic, non-radioactively labeled core material that serves as a support for a polymeric coating comprised of a linear, branched, or dendrimer biocompatible polymer to which a suitable binding agent is attached. The binding agent is selected from a number of chemically stable compounds that bind radioactive or non-radioactive therapeutic agents. The material may have attached an alpha, beta- or gamma-emitting radionuclide, or all three, depending upon the clinical need.

In order to overcome the problem of leaching of radionuclide from ceramic microspheres, while at the same time maintaining the microspheres with a low density, the present invention provides microspheres with improved characteristics arising from the fact that the microspheres can be formulated to be of such a size, shape and density that they have improved distribution characteristics when administered into the arterial supply of target organs to be treated. In addition, each microsphere can deliver a higher amount of ionising radiation than prior art microspheres. This, in turn, means that a lesser number can be administered to the target organ in order to deliver the same radiation dose. In another improvement, the microspheres can be labeled after manufacture, thereby improving the manufacture process.

The chemical durability of the microspheres is such that they do not release a significant amount of radiation emitting radioisotope into the circulatory system upon administration.

The method provides three ways of controlling the total dose delivered to a site, while simultaneously controlling exposure to other areas of the body. First, the total amount of isotope can be varied. Second, the half-life of the isotope can be selected; this provides an upper limit of the applied dose. Third, the lifetime of the radioisotope in the local delivery depot can be controlled.

An aspect of the invention relates to a method for embolization including delivery of an embolic agent composition to a blood vessel to fill or plug the blood vessel and/or encourage clot formation so that blood flow through the vessel is reduced or stopped.

The present invention is also directed towards a therapeutic radioisotope effector combined with an imaging radioisotope effector where the therapeutic radionuclide and the imaging or diagnostic radionuclide resides on the same microparticle carrier.

Furthermore, the present invention is directed towards targeted microparticle constructs where both yttrium-90 and indium-111 or a technetium isotope may be combined in the same microparticle constructs.

The present invention also provides a method of radiation therapy of a human or other mammalian patient, which comprises administration to the patient of a radiation emitting radionuclide.

Preferably, the beta-radiation emitting radionuclide is yttrium-90.

Preferably, the therapy comprises treatment of cancer or tumours, particularly primary or secondary cancer of the liver, in the patient.

To ensure clarity of the description that follows, the following definitions are provided.

By “microparticles” or “microspheres” is meant particles that support an effector substance over its surface. The microparticle is non-biodegradable and biocompatible.

By “non-biodegradable” is meant a material that should not degrade by bodily processes to a significant extent over the period of therapy.

By “biocompatible” is meant not toxic to the body, is pharmaceutically acceptable, is not carcinogenic, and does not significantly induce inflammation in body tissues.

A “linking carrier”, as used herein, is a molecule that is used to join the effector molecule to the microparticle. The linker is capable of forming covalent bonds to both the effector and the microparticle matrix.

An “effector” is a molecular construct that may involve a chelator that carries out a useful biological function within the body. As used herein, the term therapeutic effector is used to mean any compound or molecule or isotope that will either cause, elicit or initiate a cellular or physiological response within the targeted tissue.

A “chelator” or “bonding unit” is the moiety or group on a reagent that binds to a molecular such as a metal ion through the formation of chemical bonds with one or more donor atoms.

As used herein, “body” preferably refers to the human body, but it should be understood that body can also refer to a non-human animal body.

Microparticulate Core

The inventive augmentation material comprises smooth rounded, substantially spherical, particles of a matrix material, preferably of a biocompatible polymer. The term “substantially spherical” refers to the fact that while some of the present particles may be spheres, most of the particles of the present invention are sphere-like in their shape, i.e., they are spheroidal. The terms “rounded” or “smooth, rounded” as used herein refers to the fact even though the present particles are not perfect spheres, they do not have any sharp or angular edges. The particles must be sufficiently large so as to avoid phagocytosis.

As used herein, the term “microparticles” refers to particles having a number median diameter of greater than 7 microns. In a particular embodiment, the microparticles have a number median diameter of greater than 10 microns. For example, the core diameter may be from about 10 microns to about 200 microns. Preferably also, the microspheres have a diameter in the range of from 20 to 80 microns.

However, it is understood that for introduction by injection the upper limit on particle size will be dictated by the particular injection equipment employed. That is, the particles must be sufficiently small so as to avoid aggregation and clogging of the syringe when being injected. A typical range for injection is from about 10 to 150 microns, preferably in a narrow particle size range extending not more than about 35 microns, and more preferably extending not more than about 20 to 30 microns, and most preferably having substantially equivalent particle sizes.

The microparticle diameter may be from about 10 microns to about 200 microns. In one embodiment, the microspheres have a diameter in the range of from 8-100. In another embodiment, the microspheres have a diameter of from 20 to 30 microns.

These are meant to be exemplary and not limiting. Other narrow particle size ranges within the overall size range of 10 to 150 microns can also be used. In discussing these ranges, it should be understood that as a practical matter, a small amount of particles outside the desired range may be present in a sample of the present augmentation material. However, most of the particles in any given sample should be within the desired range. Preferably, 90% of the particles are within the desired range and most preferably 95-99% are within the range.

As used herein, the term “particle size” refers to a number median diameter as determined by conventional particle size measuring techniques known to those skilled in the art, such as, laser diffraction, photon correlation spectroscopy, sedimentation field flow fractionation, disk centrifugation or electrical sensing zone method. Laser diffraction is preferred. The “number median diameter” reflects the distribution of particles (by number) as a function of particle diameter. An alternative designation of particle size often used in the art is the “volume median diameter”. The volume median diameter is the median diameter of the volume weighted size distribution. The volume median diameter reflects the distribution of volume as a fiction of particle diameter.

In a preferred embodiment, the microparticle has a diameter which is selected to lodge in particular regions of the body. Use of microspheres that lodge within organs or regions is known in studies of blood flow (Flaim et al, J Pharmacol. Meth. 11:1-39, 1984; Heymann et al, Prog. Cardiovasc. Dis. 20:55-79, 1977). For example, a microparticle selected to lodge in a capillary will typically have a diameter most preferably between 15 to 35 microns. Microparticles can be fabricated from different polymers using a variety of different methods known to those skilled in the art. Numerous methods are known for preparing microparticles of any particular size range. Synthetic methods for microparticles from molten materials, are known, and include polymerization in emulsion, in sprayed drops, and in separated phases. For solid materials or preformed gels, known methods include wet or dry milling or grinding, pulverization, classification by air jet or sieve, and the like.

A further preferred feature of the particulate material of the present invention is that the microspheres have a density in the range of from 1 to 4 gm/cm³, more preferably in the range of from 1 to 2 gm/cm³.

In the present invention, locally deposited polymer depots on the surface of the microsphere core are used as a vehicle for the immobilization and local delivery of a radionuclide or radiopharmaceutical.

The interior of said core preferably does not contain radioactive therapeutic agent.

In one embodiment, the microparticle is not water swellable.

Biocompatible Microparticulate Core Materials

The preferred microparticulate cores of the invention are polymers that are biocompatible. Suitable biocompatible polymers can be either slowly biodegradable or non-biodegradable polymers or blends or copolymers thereof, as described herein. The biocompatible polymers suitable for use in the invention can therefore be water-insoluble or minimally water-soluble.

A polymer is biocompatible if the polymer and any degradation products of the polymer are non-toxic to the recipient and also possess no significant deleterious or untoward effects on the recipient's body, such as an immunological reaction at the injection site.

Suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl flouride, poly(vinyl imidazole), chlorosulphonate polyolefins, polyethylene oxide, blends thereof, and copolymers thereof.

Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Synthetically modified natural polymers include alkyl celluloses, hydroxyalkyl celluloses, cellulose ethers, cellulose esters, and nitrocelluloses. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

Suitable polymer compositions preferably have intrinsic and controllable biodegradability, so that they persist for about a week to about six months; are non-toxic, containing no significant toxic monomers and degrading into non-toxic components; are biocompatible; are chemically compatible with the substances to be delivered, are able to remain at the site of application by adherence or by geometric factors, such as by being trapped at a desired location; are capable of being delivered by techniques of minimum invasivity, such as by catheter.

Acceptable molecular weights for polymers used in this invention can be determined by a person of ordinary skill in the art taking into consideration factors such as the desired polymer degradation rate, physical properties such as mechanical strength, and rate of dissolution of polymer in solvent. Typically, an acceptable range of molecular weight is of about 2,000 Daltons to about 2,000,000 Daltons. (Polymer molecular weights are usually represented as weight average molecular weights. However, for dendrimers the reported molecular weights are absolute as they have a defined chemistry.)

In one embodiment, the biocompatible polymer core and the biocompatible polymer of said linking carrier comprise different biocompatible polymers.

Linking Carriers

Preferred linking carriers are biocompatible polymers (such as HPMA), macromolecular assemblies of biocompatible components (such as polymeric dendrimers), or multi-component linking carriers consisting of more than one biocompatible component (such as dendrimer-coated polymeric microparticles).

Examples of linking carriers include, but are not limited to, polymerized copolymers, dendrimers, polyethylene glycol assemblies, capped polylysines, poly(hydroxybutyric acid), dextrans, biocompatible polymers and copolymers such as hyaluronic acids and acrylamides and derivatives thereof, and polystyrene particles and derivatives thereof. A preferred linking carrier is a dendrimer.

The linking carrier can be coupled to the effector by a variety of methods, depending on the specific chemistry involved. The coupling will be covalent. A variety of methods suitable for coupling of the targeting entity and the therapeutic effector to the linking carrier can be found in Hemanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the effector. Large numbers of effectors may be attached to one microparticle.

Water-soluble polymers (dendrimers, PEG etc) may be selected as a biocompatible linker in order to avoid immunogenic responses upon administration.

Dendrimer Linking Carriers

Another preferred linking carrier is a dendrimer. Dendrimers are polymers with well-defined branching from a central core (e.g., “starburst polymers”). In contrast to conventional polymers, dendrimers tend to be highly branched macromolecules. Dendrimers are described in U.S. Pat. Nos. 4,507,466, 4,558,120, 4,568,737, 4,587,329, 4,631,337, 4,694,064, 4,737,550, and 4,857,599, as well as numerous other patents and patent publications. Dendrimer structure, synthesis, and characteristics are reviewed in Kim and Zimmerman, “Applications of dendrimers in bio-organic chemistry,” Current Opinion In Chemical Biology (1998) 2(6):733-42; Tam and Spetzler, “Chemoselective approaches to the preparation of peptide dendrimers and branched artificial proteins using unprotected peptides as building blocks,” Biomedical Peptides, Proteins & Nucleic Acids (1995) 1(3):123-32; Frechet, “Functional polymers and dendrimers: reactivity, molecular architecture, and interfacial energy,” Science (1994) 263(5154):1710-5; Liu and Frechet, “Designing dendrimers for drug delivery,” Pharmaceutical Science and Technology Today (1999) 2(10):393401; Verprek and Jezek “Peptide and glycopeptide dendrimers. Part I,” Journal of Peptide Science (1999) 5(1):5-23; Veprek and Jezek, “Peptide and glycopeptide dendrimers. Part II,” Journal Of Peptide Science (1999) 5(5)203-20; Tomalia et al., “Starburst dendrimers: Molecular-level control of size, shape, surface chemistry, topology, and flexibility from atoms to macroscopic matter” Angewandte Chemie—International Edition in English (1990) 29(2):138-175; Bosman et al., “About dendrimers: Structure, physical properties, and applications” Chemical Reviews (1999) 99(7):1665-1688; Fischer and Vogtle, “Dendrimers: From design to application—A progress report,” Angewandte Chemie-International Edition (1999) 38(7):885905; Roovers and Comanita, “Dendrimers And Dendrimer-Polymer Hybrids,” Advances In Polymer Science (1999) 142:179-228; Smith and Diederich, “Functional Dendrimers: Unique Biological Mimics,” Chemistry—A European Journal (1998) 4(8):1353-1361; and Matthews et al., “Dendrimers—Branching out from curiosities into new technologies,” Progress In Polymer Science (1998) 23(1): 1-56. The synthesis of dendrimers typically uses reiterative synthetic cycles, allowing control over the dendrimer's size, shape, surface chemistry, flexibility, and interior topology. An example of a dendrimer suitable for use as a linking entity is described in Wu et al., “Metal-Chelate-Dendrimer-Antibody Constructs for Use in Radioimmunotherapy and Imaging,” Bioorganic and Medicinal Chemistry Letters (1994) 4(3):449-454.

Dendrimers can be readily used as linking carriers by employing a variety of chemical conjugation techniques to attach the targeting entity and therapeutic entity. For example, in U.S. Pat. No. 6,020,457, which discloses a dendrimer having a disulfide (—S—S—) bond in its core, the dendrimer can be constructed by the methods described in the patent. The final external layer of the dendrimer can be capped with a reactive group such as an amine or carboxyl group. These reactive groups can then be derivatized with either targeting entities or therapeutic entities (or, in some cases, a mixture of both).

A dendrimer for the purposes of the present invention is a branched polymer which is a three-dimensional highly ordered compound, in which branched oligomeric/polymeric sequences may be formed around a nuclear molecule by reiterative reaction sequences, and which under certain conditions has a positively charged outer surface as a result of suitable functional terminal end groups (polycationic dendrimer). Dendrimers of this kind and their preparation are described in WO 84/02705, U.S. Pat. Nos. 4,507,466, 4,558,120, 4,568,737, 4,587,329, 4,631,337, 4,694,064, 4,713,975, 4,737,550, 4,871,779, 4,857,599, EP 0 234 408, EP 0 247 629, EP 0 271 180, and especially Tange et al., supra, WO 95/02397, and Tomalia et al., supra.

Dendrimers which are suitable for the present invention include, for example, polyamidoamine (PAMAM) dendrimers which may be synthesised around ammonia, tris-(2-aminoethyl)amine (TAEA) or ethylenediamine (EDA) as nuclear molecules by stepwise addition of the two monomers methacrylate and ethylenediamine (Tang et al., supra). The terminal groups of such a dendrimer are preferably primary amino groups. 5th, 6th or 7th generation PAMAM dendrimers are preferred, particularly 6th generation, according to Tang et al., supra. The theoretical molecular weights, number of terminal amines and hydrodynamic radii of such PAMAM dendrimers may be found in the publication of Tang et al., supra. Table 1 shows the properties of amine functional PAMAM dendrimers.

TABLE 1

| Catalog | Generation | Molecular | Diameter | No. of surface |
|---|---|---|---|---|
| No. | No. | weight (Da) | (Å) | amino groups |
| 41,236-8 | 0 | 517 | 15 | 4 |
| 41,238-4 | 1 | 1430 | 22 | 8 |
| 41,240-6 | 2 | 3256 | 29 | 16 |
| 41,242-2 | 3 | 6909 | 36 | 32 |
| 41,244-9 | 4 | 14215 | 45 | 64 |
| 53,670-9 | 5 | 28826 | 54 | 128 |
| 53,671-7 | 6 | 58048 | 67 | 256 |
| 53,672-5 | 7 | 116493 | 81 | 512 |
| 53,674-1 | 8 | 233383 | 97 | 1024 |
| 53,676-8 | 9 | 467162 | 114 | 2048 |
| 53,677-6 | 10 | 934720 | 135 | 4096 |

The dendritic polymers which may be used include generally any of the known dendritic architectures including dendrimers, regular dendrons, controlled hyperbranched polymers, dendrigrafts, and random hyperbranched polymers. Dendritic polymers are polymers with densely branched structures having a large number of reactive groups. A dendritic polymer includes several layers or generations of repeating units which all contain one or more branch points. Dendritic polymers, including dendrimers and hyperbranched polymers, are prepared by condensation reactions of monomeric units having at least two reactive groups. The dendrimers which can be used include those comprised of a plurality of dendrons that emanate from a common core which can be a single atom or a group of atoms. Each dendron generally consists of terminal surface groups, interior branch junctures having branching functionalities greater than or equal to two, and divalent connectors that covalently connect neighboring branching junctures.

The hyperbranched polymers which may be used represent a class of dendritic polymers which contain high levels of nonideal irregular branching as compared with the more nearly perfect regular structure of dendrons and dendrimers. Specifically, hyperbranched polymers contain a relatively high number of irregular branching areas in which not every repeat unit contains a branch juncture. The preparation and characterization of dendrimers, dendrons, random hyperbranched polymers, controlled hyperbranched polymers, and dendrigrafts is well known. Examples of dendimers and dendrons, and methods of synthesizing the same are set forth in U.S. Pat. Nos. 4,410,688, 4,507,466; 4,558,120; 4,568,737; 4,587,329; 4,631,337; 4,694,064; 4,713,975; 4,737,550; 4,871,779 and 4,857,599. Examples of hyperbranched polymers and methods of preparing the same are set forth, for example in U.S. Pat. No. 5,418,301.

Dendritic polymers suitable for use with the invention also include macromolecules commonly referred to as cascade molecules, arborols, arborescent grafted molecules, and the like. Suitable dendritic polymers also include bridged dendritic polymers, i.e., dendritic macromolecules linked together either through surface fumctional groups or through a linking molecule connecting surface functional groups together, and dendritic polymer aggregates held together by physical forces. Also included are spherical-shaped dendritic polymers and rod-shaped dendritic polymers grown from a polymeric core.

U.S. Pat. No. 5,338,532 teaches polymer conjugates comprising dense star polymers associated with a carried material, the disclosure of which is hereby incorporated by reference. (One type of dense star polymers is Starburs™ polymers (trademark of The Dow Chemical Company) where the dendrimer is a polyamidoamine (PAMAM).) A variety of suitable applications for such conjugates are broadly discussed in U.S. Pat. No. 5,338,532, including the use of these conjugates as delivery vehicles for biologically active agents. U.S. Pat. No. 5,338,532 exemplifies the use of zero valence metals, and ionic or radioactive metals, specifically exemplifying Fe, Rh, Pd, Y, Fn, Pb, Gd, Mn and Gd.

Dendritic polymers suitable for use with the present invention also include macromolecules commonly referred to as cascade molecules (e.g., E. Buhleier et al., Synthesis 155-158 (February 1978), arborols (e.g., U.S. Pat. Nos. 5,376,690 and 5,210,309), arborescent grafted molecules, tectodendrimers (e.g., Srinivas Uppuluri et al., “Tecto(dendrimer) Core-shell Molecules: Macromolecular Tectonics for the Systematic Synthesis of Larger Controlled Structure Molecules” PMSE, Spring Meeting (Mar. 21-25, 1999) 55-56), and the like. Suitable dendritic polymers also include bridged dendritic polymers, i.e., dendritic macromolecules linked together either through surface functional groups or through a linking molecule connecting surface functional groups together, and dendritic polymer aggregates held together by physical forces. Also included are spherical-shaped dendritic polymers (e.g., U.S. Pat. Nos. 4,507,466; 4,588,120; 4,568,737; 4,631,337; 4,587,329; and 4,737,550, the disclosures of which are hereby incorporated by reference) and rod-shaped dendritic polymers (e.g., U.S. Pat. No. 4,694,064, the disclosure of which is hereby incorporated by reference) grown from a polymeric core. Additional dendritic polymers suitable for use with the present invention include all the basic dendritic structures where specific chelating groups or moieties are either in the central core of the dendrimer, and/or located within the interior structure on the dendron arms and/or located on the surface of the dendrimer. All of these above dendrimer terms are to be understood to be included within the term “dendritic polymer.”

Dendritic polymers which are useful in the practice of this invention include those that have symmetrical branch cells (arms of equal length, e.g., PAMAM dendrimers; for example described in U.S. Pat. No. 5,527,524) and those having unsymmetrical branch cells (arms of unequal length, e.g. lysine-branched dendrimers, for example described in U.S. Pat. No. 4,410,688), branched dendrimers, cascade molecules (e.g., E. Buhleier et al., Synthesis 155-158 (February 1978)), arborols (e.g., U.S. Pat. Nos. 5,376,690 and 5,210,309), and the like.

The dendritic polymers used in the practice of this invention can be generationally monodisperse or generationally polydisperse. Dendritic polymers in a monodisperse solution are substantially all of the same generation, and hence of uniform size and shape. The dendritic polymers in the polydisperse solution comprise a distribution of different generation polymers. The dendritic polymer molecules which may be used in the practice of this invention include mixtures of different interior and exterior compositions or functionalities. Examples of suitable dendritic polymers include poly(ether) dendrons, dendrimers and hyperbranched polymers, poly(ester) dendrons, dendrimers and hyperbranched polymers, poly(thioether) dendrons, dendrimers and hyperbranched polymers, poly(amino acid) dendrons dendrimers and hyperbranched polymers, poly(arylalkylene ether) dendritic polymers and polypropylamine dendrimers, dendrimers and hyperbranched polymers. Poly(amidoamine) (PAMAM) dendrimers have been found to be particularly useful for preparing the metal-containing complexes of this invention.

The dendritic polymers which are believed to be most useful in the practice of this invention are approximately monodispersed. That is, dendritic polymers in a monodispersed solution in which all of the dendritic polymer molecules are substantially of the same generation, and hence of uniform size and shape, are preferred. Monodispersed solutions of dendrimers are particularly preferred.

The dendritic polymers preferred for use in the practice of this invention have terminal functional groups which are accessible to a chelate containing compound which is capable of interacting with the functional groups.

The term “functional group” is intended to comprise groups such as e.g. ester groups, ether groups, thiol groups, carbonyl groups, hydroxyl groups, amide groups, carboxylic groups, and imide groups as well as combinations thereof. Amine-terminated polyamidoamine, polyethyleneimine and polypropyleneimine dendrimers are also known, for example, from U.S. Pat. Nos. 5,393,797; 5,393,795; 5,560,929; and 5,387,617, all to Hedstrand et al.

The optional linking dendrimers may be incorporated to increase the polyvalency of yttrium attachment sites. The microsphere surface may already contain multiple sites to attach chelator for yttrium. However since the surface of a microsphere may be rigid, chemical modification is a difficult reaction. Hence linkers may be needed to increase the distance from the sphere surface to facilitate reaction with the chelator. The linker may be attached if suboptimal concentrations of chelator are obtained on the surface of the spheres.

Dendrimer Size

Dendrimers are generally prepared by stepwise or reiterative reaction of multifunctional monomers to obtain a branched structure. In U.S. Pat. No. 5,530,092, for example, the repetition of double Michael addition of acrylonitrile starting with a primary diamine followed by hydrogenation obtains two primary amines for each initial amine. This doubles the number of primary amine groups. Thus, beginning with a diamine, the first generation dendrimer (G1) has four primary amines; the second generation (G2) has eight primary amines; the third generation (G3) has sixteen primary amines; the fourth generation (G4) has thirty-two primary amines; the fifth generation (G5) has sixty-four primary amines in the outer shell, and so on. These polyamine dendrimers are said to be stable to degradation through hydrolysis reactions.

The generation of the dendritic polymer, and hence the size of the dendritic polymer, which may be utilized in the practice of this invention may vary considerably. For example, generation 3.5 poly(amidoamine) dendrimers (3.5 PAMAM) are acceptable for use in the practice of this invention. However, higher and lower generations are also expected to be useful, but especially the range from generation 3.5 to 7.5 for PAMAM dendrimers having an ethylenediamine (EDA) core.

Methods of Coupling to the Linking Carrier

It is intended to covalently attach an effector to the linking carrier. This covalent attachment may be directly between the surface of the linking carrier and the effector by means of a linker moiety between the surface of the linking carrier and the effector. Some linkers that may be used are described in U.S. Pat. No. 5,527,524; EP 0353450; EP 0570575; and EP 0296522, the disclosures of which are hereby incorporated by reference.

Generally, prior to forming the linkage between the linking carrier and the effector, and optionally, the spacer group, at least one of the chemical functionalities will be activated. One skilled in the art will appreciate that a variety of chemical functionalities, including hydroxy, amino, and carboxy groups, can be activated using a variety of standard methods and conditions.

Typically, the agent is linked covalently to a linking carrier using standard chemical techniques through their respective chemical functionalities. Optionally, the linking carrier or agent is coupled to the agent through one or more spacer groups. The spacer groups can be equivalent or different when used in combination. Likewise, if more than one linking carrier is used to produce the agent- linking carrier complex, the dendrimers can be equivalent or different.

In certain embodiments, one or more of the active groups are protected during one or more steps of the reaction to assemble the linking carrier or a conjugate of the linking carrier. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

The effector molecule may be attached to the linking carrier by any of a number of means well known to those of skill in the art. Typically the effector is conjugated, either directly or through a dendrimer or other linker (spacer), to the microparticle.

Alternatively, the microparticle and/or spacer may be derivatized to expose or attach additional reactive functional groups. The derivatization may involve attachment of any of a number of linker molecules such as those available from Pierce Chemical Company, Rockford Ill.

A bifunctional linker having two functional groups reactive with a group on a particular effector may be used to form the desired conjugate.

Many procedures and linker molecules for attachment of various compounds including radionuclide metal chelates, toxins and drugs to proteins such as antibodies are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075).

Spacer Groups

One or more spacer groups optionally may be introduced between the linking carrier and the therapeutic agent. Spacer groups contain at least two chemical functionalities. Typically, one chemical functionality of the spacer group bonds to a chemical functionality of the linking carrier, while the other chemical functionality of the spacer group is used to bond to a chemical functionality of the pharmaceutical agent. Examples of chemical functionalities of spacer groups include hydroxy, mercapto, carbonyl, carboxy, amino, ketone, and mercapto groups. Spacer groups may also be used in combination. When a combination of spacer groups is used, the spacer groups may be different or equivalent. The spacer group is an optional moiety which may be introduced to increase the length of the linker so that it is spaced further away from the microsphere surface thereby providing flexibility and facilitate reaction with the chelator for the radionuclide (i.e., yttrium).

Whether coupled directly, or through a spacer, the agent is preferably coupled to the linking carrier via a covalent bond. The covalent bond may be non-reversible, partially reversible, or fuilly reversible. The degree of reversibility corresponds to the susceptibility of the agent- linking carrier complex to in vivo degradation. As will be apparent to those of skill in the art, such reversible groups can be incorporated at any point within the linking carrier -agent conjugate. The introduction of a spacer arm having a reversible linkage is merely an exemplary embodiment; the bond between the agent and the dendrimer, for example, may also be reversible.

The susceptibility of the agent- linking carrier complexes to degradation can be ascertained through studies of the hydrolytic or enzymatic conversion of the complex to the unbound pharmaceutical agent. Generally, good correlation between in vitro and in vivo activity is found using this method. See, e.g., Phipps et al., J. Pharm. Sciences 78:365 (1989). The rates of conversion may be readily determined, for example by spectrophotometric methods or by gas-liquid or high pressure liquid chromatography. Half-lives and other kinetic parameters may then be calculated using standard techniques. See, e.g., Lowry et al. MECHANISM AND THEORY IN ORGANIC CHEMISTRY, 2nd Ed., Harper & Row, Publishers, New York (1981).

Effectors

The linking carrier may be conjugated to a variety of effectors useful for treating or identifying diseased tissue. Preferably the effectors that are conjugated to the polymer conjugate are selected from the group consisting of therapeutic or diagnostic agents.

Examples of therapeutic agents for use with the invention include, but are not limited to, metal chelate complexes, drugs, prodrugs, radionuclides, boron addends, labeling compounds, toxins and other effector molecules, such as cytokines, lymphokines, chemokines, immunomodulators, radiosensitizers, asparaginase, boron addends and radioactive halogens. Preferably, the therapeutic agent that is conjugated to the polymer backbone is selected from the group consisting of therapeutic radioisotpes, toxins, drugs, prodrugs and boron addends.

Drugs for use with the current invention include, but are not limited to, any currently approved or not-yet-approved chemotherapy drug, as long as it can be attached to the polymer conjugate. Typically useful already approved drugs include, but are not limited to, the following agents and derivatives of these agents: anastrozole, azacytidine, bleomycin, busulfan, carboplatin, carmustine, chlorambucil, cisplatin, cladribine, cyclophosphamide, cytarabine, dacarbazine, dactinomycin, daunorubicin, docetaxel, doxorubicin, estramustine, etoposide, floxuridine, fludarabine, fluorouracil, flutamide, gemcitabine, hydroxyurea, idarubicin, ifosfamide, irinotecan, lomustine, mechlorethamine, megestrol, melphalan, mercaptopurine, methotrexate, mitomycin, mitotane, mitoxantrone, paclitaxel, pentostatin, procarbazine, tamoxifen, teniposide, thioguanine, thiotepa, topotecan, vinblastine, vincristine, and vinorelbine.

Additionally, the polymer conjugate may comprise a therapeutic agent consisting of boron addends to be used in Boron Neutron Capture Therapy (BNCT) protocols. BNCT is a binary system designed to deliver ionizing radiation to tumor cells by neutron irradiation of tumor-localized boron-10 atoms. BNCT is based on the nuclear reaction which occurs when a stable isotope, isotopically enriched B-10 (present in 19.8% natural abundance), is irradiated with thermal neutrons to produce an alpha particle and a Li-7 nucleus. These particles have a path length of about one cell diameter, resulting in high linear energy transfer. Just a few of the short-range 1.7 MeV alpha particles produced in this nuclear reaction are sufficient to target the cell nucleus and destroy it. Success with BNCT of cancer requires methods for localizing a high concentration of boron-10 at tumor sites, while leaving non-target organs essentially boron-free. Compositions and methods for treating tumors in patients using pre-targeting msAb for BNCT are described in U.S. Pat. No. 6,228,362 and can easily be modified in accordance with the present invention, and is hereby incorporated by reference. Additionally, other elements are suitable for neutron capture reactions. One example is uranium. Uranium, in large amounts, can be bound by naturally occurring chelating agents such as ferritin. Such strategies have been described in the art, for example U.S. Pat. No. 6,228,362 and references cited therein are easily adaptable to the present invention and are hereby incorporated in their entirety by reference.

The embodiment of the invention in which the diagnostic agent is a contrast agent is illustrated by reference to metal chelate-based contrast agents. The focus on metal chelates is intended as illustrative rather than limiting. Those of skill in the art will appreciate that many contrast agents other than metal chelates can be conjugated to the linking carrier of the invention (e.g. particles, iodinated aryl compounds, nitroxides, etc.).

In preferred embodiments, the therapeutic metal ion is associated with the microparticle construct via a chelator.

An array of metal chelates is known in the art. See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

In a preferred embodiment, the diagnostic agent is a metal complex of polyaminocarboxylate chelating agent such as diethylenetriaminepentaacetic acid (DTPA).

In other preferred embodiments, the therapeutic entity is a chemotherapeutic agent or prodrug or toxin where the therapeutic entity is attached to the surface of the linking carrier. Alternatively, the therapeutic entity may be entrapped or encapsulated within the linking carrier.

In a particularly preferred embodiment, the therapeutic radionuclide is associated with a chelator that is chemically attached to a polymeric surface in the microparticle construct. In another particularly preferred embodiment, yttrium-90 is the therapeutic radionuclide, and DOTA is the chelator.

Chelating Groups

Chelating groups are well known to those of skill in the art. Wu et al. (1992) Nucl. Med. Biol., 19(2): 239-244 discloses a synthesis of macrocylic chelating agents for radiolabeling proteins with ¹¹¹In and ⁹⁰Y.

Preferred water soluble chelators to be used in the practice of the present invention include, but are not limited to, diethylenetriaminepentaacetic acid (DTPA), ethylenediaminetetraacetic acid (EDTA), 1,4,7,10-tetraazacyclododecane-N,N′, N,″N′″ tetraacetate (DOTA), tetraazacyclotetradecane-N,N″, N″N″-tetraacetic acid (TETA), cyclohexyl 1,2-diamine tetra-acetic acid (CDTA), ethyleneglycol-O,O′-bis(-2-aminoethyl)-N,N,N′,N′-tetra-acetic acid (EGTA), N,N-bis(hydroxybenzyl)-e-thylenediamine-N,N′-diacetic acid (HBED), triethylene tetramine hexa-acetic acid (TTHA), hydroxyethyldiamine triacetic acid (HEDTA), hydroxyethylidene diphosphonate (HEDP), dimercaptosuccinic acid (DMSA), diethylenetriaminetetramethylenephosphonic acid (DTTP) and 1-(p-aminobenzyl)-DTPA, 1,6-diamino hexane-N,N,N′,N′-tetraacetic acid, DPDP, ethylenebis (oxyethylenenitrilo)-tetraacetic acid, and cyclohexyldiethylenetriaminepentaacetic acid ligand (CHX-DTPA).

One chelating agent, 1,4,7,10-tetraazacyclododecane-N, N, N″, N′″-tetraacetic acid (DOTA), is of particular interest because of its ability to chelate a number of diagnostically and therapeutically important metals, such as radionuclides and radio labels.

In certain embodiments of the present invention, DOTA or other chelating agent conjugates, such as EDTA, or DTPA, for example, may be prepared in the form of water-soluble salts (sodium salt, potassium salt, tetrabutylammonium salt, calcium salt, ferric salt, etc.). These salts will be useful as therapeutic agents for tumor treatment. Secondly, DTPA or other chelating agents will be useful as diagnostic agents which when labeled with radionuclides such as ¹¹¹In or ^{99m}Tc, may be used as radiotracers to detect certain tumors in combination with nuclear imaging techniques.

In some embodiments, the chelator contains or is a derivative of 1,4,7,10-tetraazacyclododecane-N,N′,N″,N′″-tetraacetic acid (DOTA) or the chelators known to those skilled in the art. In other embodiments, the chelator comprises an ionizable group such as carboxyl, phosphate, phosphonate, sulfate, sulfonate, or sulfinate. In still other embodiments, the chelator comprises a single ionizable group, said single ionizable group generating a surface capable of binding an isotope or metal with a valency of +2 or greater, or +3 or greater.

As modifications and changes may be made in the structure of the water soluble polymer or a water soluble metal chelator, of the present invention and still obtain molecules having like or otherwise desirable characteristics, such “biologically functional equivalents” or “functional equivalents” are also encompassed within the present invention.

The nature of medical applications imposes multiple requirements on the chemical characteristics of a potential chelating ligand. It has to be (a) strong (multidentate) complexing agent for the metal ion, (b) hydrophilic to afford solubility in water, (c) nontoxic, (d) capable of incorporating into a protein structure without causing its denaturation. For virtually every single radionuclide one has to design a special chelating system. For example macrocyclic bifunctional chelating agents, in particular, DOTA, derivatives incorporating yttrium-90 and indium-111 have shown excellent kinetic stability under physiological conditions. However, the slow formation of yttrium-DOTA complexes presents a technical problem that can lead to low radiolabeling yields unless conditions are carefully controlled.

The most successful chelating agents for preparation of radioconjugates are the products of a complex organic synthesis. Quite representative examples of a synthetic procedure can be found in Brechbiel, M. W.; Gansow, O. A.; Atcher, R. W.; Schlom, J.; Esteban, J.; Simpson, D. E.; Colcher, D., Synthesis of 1-(P-isothiocyanatobenzyl) Derivatives of DTPA and EDTA. Antibody Labeling and Tumor Imaging Studies, Inorg. Chem., 1986, 25, 2772-2781. Other chelators preferred for use with this invention include the class of heterocyclic chelators

As used herein, the term “heterocycle” or “heterocyclic system” is intended to mean a stable 5, 6, or 7-membered monocyclic or bicyclic or 7, 8, 9, or 10-membered bicyclic heterocyclic ring which is saturated, partially unsaturated or unsaturated (aromatic), and which consists of carbon atoms and 1, 2, 3, or 4 heteroatoms independently selected from the group consisting of N, NH, O and S and including any bicyclic group in which any of the above-defined heterocyclic rings is fused to a benzene ring. The nitrogen and sulfur heteroatoms may optionally be oxidized.

Radionuclides

Microparticles of this invention may contain, via chelate attachment to the linking carrier, any or all of the following: phosphorus, yttrium, rhenium, and other beta emitting isotopes; actinium, bismuth, astatine and other alpha emitting isotopes; technetium, indium, iodine and other gamma emitting isotopes; and carbon, nitrogen, fluorine, sodium, magnesium, aluminum, silicon, potassium, vanadium, manganese, gallium, niobium, iodine and/or lead.

As used herein, a therapeutic radionuclide is a nuclide which undergoes spontaneous transformation (nuclear decay) with an energy transfer sufficient to impart cytotoxic amounts of radiant energy to nearby cells. In contrast, radionuclides useful for diagnosis emit radiation capable of penetrating tissue with minimal cell damage. Such radiation may be detected using a suitable scintigraphic imager. Therapeutic radionuclides of the present invention include, but are not limited to Y-90, Bi-213, At-211, I-123, I-125, I-131, At-211, Cu-67, Sc-47, Ga-67, Rh-105, Pr-142, Nd-147, Pm-151, Sm-153, Ho-166, Gd-159, Th-161, Eu-152, Er-171, Re-186, and Re-188. Diagnostic or imaging nuclides of the present invention include, but are not limited to Tc-99m, In-111, Ga-67, Rh-105, I-123, Nd-147, Pm-151, Sm-153, Gd-159, Tb-161, Er-171, Re-186, Re-188, and Tl-201.

Any useful nuclide may be used within the scope of the invention. Particularly preferred are radionuclides that have useful diagnostic or therapeutic properties, such as indium-111 or yttrium-90, respectively.

Tight binding of radiometallic nuclides often requires a chelating agent for the radiometal. Standard radiolabeling methods and precautions used in the radiolabeling of low molecular weight chelates may be used to prepare radiolabeled chelate polymers. For instance, procedures using radiometals, such as indium-111 and yttrium-90, generally require highly pure supplies of the radionuclide, deionized water in all buffer solutions, and acid-washing of glassware and plastic-ware used with any of the reagents during the radiolabeling procedures.

Procedures using radiometals such as rhenium-188, which require a chemical reduction step to effect labeling, are best carried out using oxygen-depleted buffers and argon atmospheres overlaying the radiolabeling reactions.

The coordination sphere of the radionuclide includes all the ligands or groups bound to the radionuclide. For a transition metal radionuclide, Mₜ, to be stable it typically has a coordination number (number of donor atoms) comprised of an integer greater than or equal to 4 and less than or equal to 9; that is there are 4 to 9 atoms bound to the metal and it is said to have a complete coordination sphere. The requisite coordination number for a stable radionuclide complex is determined by the identity of the radionuclide, its oxidation state, and the type of donor atoms.

Kits

The present invention also includes a kit. A “kit” comprises a collection of components, termed the formulation, in one or more vials which are used by the practicing end user in a clinical or pharmacy setting to synthesize diagnostic radiopharmaceuticals.

Where a radioactive, or other, effector is used as a diagnostic and/or therapeutic agent, it is frequently impossible to put the ready-for-use composition at the disposal of the user, because of the often poor shelf life of the radiolabelled compound and/or the short half-life of the radionuclide used. In such cases the user can carry out the labeling reaction with the radionuclide in the clinical hospital, physician's office, or laboratory. For this purpose, or other purposes, the various reaction ingredients can then be offered to the user in the form of a so-called “kit”. The kit is preferably designed so that the manipulations necessary to perform the desired reaction should be as simple as possible to enable the user to prepare from the kit the desired composition by using the facilities that are at his disposal. Therefore the invention also relates to a kit for preparing a composition according to this invention.

Such a kit according to the present invention preferably comprises a microparticle of this invention. The microparticle construct can be provided, if desired, with inert pharmaceutically acceptable carrier and/or formulating agents and/or adjuvants is/are added. In addition, the kit optionally includes a solution of a salt or chelate of a suitable radionuclide (or other active agent), and instructions for use with a prescription for administering and/or reacting the ingredients present in the kit.

The kit provides all the requisite components to synthesize and use the diagnostic radiopharmaceutical except those that are commonly available to the practicing end user, such as water or saline for injection, a solution of the radionuclide, equipment for heating the kit during the synthesis of the radiopharmaceutical, if required, equipment necessary for administering the radiopharmaceutical to the patient such as syringes and shielding, and imaging equipment.

The kit may contain a transfer ligand, a reducing agent, a lyophilization aid, a stabilization aid, a solubilization aid and bacteriostats as well as the active microparticle and bound effectors.

A “transfer ligand” is a ligand that forms an intermediate complex with a metal ion that is stable enough to prevent unwanted side-reactions but labile enough to be converted to a metallopharmaceutical. The formation of the intermediate complex is kinetically favored while the formation of the metallopharmaceutical is thermodynamically favored. Transfer ligands usefuil in the preparation of metallopharmaceuticals and in diagnostic kits useful for the preparation of diagnostic radiopharmaceuticals include but are not limited to gluconate, glucoheptonate, mannitol, glucarate, N,N,N′,N′-ethylenediaminetetraacetic acid, pyrophosphate and methylenediphosphonate. In general, transfer ligands are comprised of oxygen or nitrogen donor atoms.

A “reducing agent” is a compound that reacts with a radionuclide, which is typically obtained as a relatively unreactive, high oxidation state compound, to lower its oxidation state by transferring electron(s) to the radionuclide, thereby making it more reactive. Reducing agents useful in the preparation of radiopharmaceuticals and in diagnostic kits for the preparation of the radiopharmaceuticals include but are not limited to stannous chloride, stannous fluoride, formamidine sulfinic acid, ascorbic acid, cysteine, phosphines, and cuprous or ferrous salts. Other reducing agents are described in Brodack et. al., PCT Application 94/22496, which is incorporated herein by reference.

A “lyophilization aid” is a component that has favorable physical properties for lyophilization, such as the glass transition temperature, and is added to the formulation to improve the physical properties of the combination of all the components of the formulation for lyophilization.

Lyophilization aids useful in the preparation of diagnostic kits useful for the preparation of radiopharmaceuticals include but are not limited to mannitol, lactose, sorbitol, dextran, Ficoll, and polyvinylpyrrolidine (PVP).

A “stabilization aid” is a component that is added to the metallopharmaceutical or to the diagnostic kit either to stabilize the metallopharmaceutical or to prolong the shelf-life of the kit before it must be used. Stabilization aids can be antioxidants, reducing agents or radical scavengers and can provide improved stability by reacting preferentially with species that degrade other components or the metallopharmaceutical.

Stabilization aids useful in the preparation of metallopharmaceuticals and in diagnostic kits useful for the preparation of radiopharmaceuticals include but are not limited to ascorbic acid, cysteine, monothioglycerol, sodium bisulfite, sodium metabisulfite, gentisic acid, and inositol.

A “solubilization aid” is a component that improves the solubility of one or more other components in the medium required for the formulation.

Solubilization aids useful in the preparation of metallopharmaceuticals and in diagnostic kits useful for the preparation of radiopharmaceuticals include but are not limited to ethanol, glycerin, polyethylene glycol, propylene glycol, polyoxyethylene sorbitan monooleate, sorbitan monoloeate, polysorbates, poly(oxyethylene)poly(oxypropylene)poly(oxyethylene) block copolymers (Pluronics) and lecithin. Preferred solubilizing aids are polyethylene glycol, and Pluronics.

Buffers useful in the preparation of metallopharmaceuticals and in diagnostic kits useful for the preparation of said radiopharmaceuticals include but are not limited to phosphate, citrate, sulfosalicylate, and acetate. A more complete list can be found in the United States Pharmacopeia.

Bacteriostats useful in the preparation of metallopharmaceuticals and in diagnostic kits useful for the preparation of radiopharmaceuticals include but are not limited to benzyl alcohol, benzalkonium chloride, chlorbutanol, and methyl, propyl or butyl paraben.

A component in a diagnostic kit can also serve more than one fluction. A reducing agent can also serve as a stabilization aid, a buffer can also serve as a transfer ligand, a lyophilization aid can also serve as a transfer, ancillary or co-ligand and so forth.

Therapeutic radiopharmaceuticals, X-ray contrast agent pharmaceuticals, ultrasound contrast agent pharmaceuticals and metallopharmaceuticals for magnetic resonance imaging contrast are provided to the end user in their final form in a formulation contained typically in one vial, as either a lyophilized solid or an aqueous solution. The end user reconstitutes the lyophilized with water or saline and withdraws the patient dose or just withdraws the dose from the aqueous solution formulation as provided.

The technetium and rhenium radiopharmaceuticals of the present invention can be easily prepared by admixing a salt of a radionuclide, a compound of the present invention, and a reducing agent, in an aqueous solution at temperatures from 0 to 100° C. The technetium and rhenium radionuclides are preferably in the chemical form of pertechnetate or perrhenate and a pharmaceutically acceptable cation. The pertechnetate salt form is preferably sodium pertechnetate such as obtained from commercial Tc-99m generators. The amount of pertechnetate used to prepare the radiopharmaceuticals of the present invention can range from 0.1 mCi to 1 Ci, or more preferably from 1 to 200 mCi.

The amount of the compounds of the present invention used to prepare the technetium and rhenium radiopharmaceuticals of the present invention can range from 0.01 μg to 10 mg, or more preferably from 0.5 μg to 200 μg. The amount used will be dictated by the amounts of the other reactants and the identity of the radiopharmaceuticals of the present invention to be prepared.

The indium, copper, gallium, silver, palladium, rhodium, gold, platinum, bismuth, yttrium and lanthanide radiopharmaceuticals of the present invention can be easily prepared by admixing a salt of a radionuclide and a reagent of the present invention, in an aqueous solution at temperatures from 0 to 100 ° C. These radionuclides are typically obtained as a dilute aqueous solution in a mineral acid, such as hydrochloric, nitric or sulfuric acid. The radionuclides are combined with from one to about one thousand equivalents of the reagents of the present invention dissolved in aqueous solution. A buffer is typically used to maintain the pH of the reaction mixture between 3 and 10.

The total time of preparation will vary depending on the identity of the metal ion, the identities and amounts of the reactants and the procedure used for the preparation. The preparations may be complete, resulting in >80% yield of the radiopharmaceutical, in 1 minute or may require more time. If higher purity metallopharmaceuticals are needed or desired, the products can be purified by any of a number of techniques well known to those skilled in the art such as liquid chromatography, solid phase extraction, solvent extraction, dialysis or ultrafiltration.

When kit constituents are used as components for pharmaceutical administration (e.g. as an injection liquid) they should be sterile. When the constituents are provided in a dry state, the user should preferably use a sterile physiological saline solution as a solvent. If desired, the constituents may be stabilized in the conventional manner with suitable stabilizers, for example, ascorbic acid, gentisic acid or salts of these acids, or they may comprise other auxiliary agents, for example, fillers, such as glucose, lactose, mannitol, and the like.

The kit to be supplied to the user may also comprise the ingredients defined above, together with instructions for use, whereas the solution of a salt or chelate of the radionuclide, defined above, which solution has a limited shelf life, may be put to the disposal of the user separately.

The kit can optionally, additionally comprise instructions for use of the composition and/or a prescription for reacting the ingredients of the kit to form the desired products. While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Pharmaceutical Compositions

The microparticles can be delivered using a fluid carrier, which can be any biologically compatible material capable of delivering the microparticles to a desired tissue site, such as a biologically compatible suspension, solution, or other form of a fluid.

The pharmaceutical compositions of this invention are particularly useful for parenteral administration, such as intravenous administration. The compositions for administration will commonly comprise a solution of the microparticulate dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

Compositions of the present invention can also include other components such as a pharmaceutically acceptable excipient, an adjuvant, and/or a carrier. For example, compositions of the present invention can be formulated in an excipient. Examples of such excipients include water, saline, Ringer's solution, dextrose solution, mannitol, Hank's solution, and other aqueous physiologically balanced salt solutions. Excipients can also contain minor amounts of additives, such as substances that enhance isotonicity and chemical stability. The preparation of an aqueous composition that contains an active ingredient is well understood in the art. Typically, such compositions are prepared as injectables, either as liquid solutions or suspensions; solid forms suitable for solution in, or suspension in, liquid prior to injection can also be prepared.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The diagnostic radiopharmaceuticals are administered by intravenous injection, usually in saline solution, at a dose of 1 to 100 mCi, or preferably at a dose of 5 to 50 mCi. Imaging is performed using known procedures.

The therapeutic radiopharmaceuticals are administered by intravenous injection, usually in saline solution, at a dose of 0.1 to 700 mCi per 70 kg body weight, or preferably at a dose of 0.5/kg to 10 mCi/kg body weight.

For parenteral administration in an aqueous solution, for example, the solution should be suitably buffered if necessary and the liquid diluent first rendered isotonic with sufficient saline or glucose. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

For injection, the agents of the invention may be formulated in aqueous solutions, preferably in physiologically compatible buffers such as Hanks's solution, Ringer's solution, or physiological saline buffer.

Single or multiple administrations of the compositions may be administered depending on the dosage and frequency as required and tolerated by the patient. In any event, the composition should provide a sufficient quantity of the microparticles of this invention to effectively treat the patient.

Therapeutically effective amounts of the therapeutic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

Imaging Detection

In the practice of one embodiment of the invention, subsequent to administration of the diagnostic agent, imaging can be performed. Tumors can be detected in body cavities by means of directly or indirectly viewing various structures to which light is delivered and then collected. Lesions at any body site can be viewed so long as nonionizing radiation can be delivered and recaptured from these structures. For example, positron emission tomography (PET) which is a high resolution, non-invasive, imaging technique can be used with the inventive antibodies for the visualization of human disease. In PET, 511 keV gamma photons produced during positron annihilation decay are detected. Similar pre-targeting strategies for PET using Fluorine-18 and Gallium-68 have been described, respectively in U.S. Pat. No. 6,187,284 and U.S. Ser. No. 09/644,706. The methodologies described in these applications are easily adaptable to the present invention and are hereby incorporated in their entirety by reference.

Particles with multiple radionuclides

A separate embodiment of the present invention is the use of multiple radionuclides on a single microparticle. Such as a core and at least two radioactive therapeutic agents attached to said core. The at least two radioactive therapeutic agents may be independently selected from the group consisting of a therapeutic radionuclide and an imaging or diagnostic radionuclide. The at least two radioactive therapeutic agents may be independently selected from an alpha-emitting radionuclide, a beta-emitting radionuclide and/or a gamma-emitting radionuclide.

In one embodiment, the at least two radioactive therapeutic agents are a combination of a beta-emitting radionuclide and a gamma-emitting radionuclide. For example, a beta-emitting radionuclide which is therapeutic radionuclide and a gamma-emitting radionuclide which is an imaging or diagnostic radionuclide. Such as yttrium-90 as a therapeutic radionuclide and indium-111 and/or Tc-99m as an imaging or diagnostic radionuclide.

In one embodiment, the core is non-ceramic and non-radioactively labeled. The core may be a polymer such as polyacrylate, ethylene-vinyl acetate polymer, an acyl substituted cellulose acetate, polyurethane, polystyrene, polyvinylchloride, polyvinyl flouride, poly(vinyl imidazole), chlorosulphonate polyolefin, polyethylene oxide, blends thereof, and copolymers thereof, a polyphosphazine, a poly(vinyl alcohol), a polyamide, a polycarbonate, a polyalkylene, a polyacrylamide, a polyalkylene glycol, a polyalkylene oxide, a polyalkylene terephthalate, a polyvinyl ether, a polyvinyl ester, a polyvinyl halide, polyvinylpyrrolidone, a polyglycolide, a polysiloxane, and copolymers thereof, a alkyl cellulose, an hydroxyalkyl cellulose, a cellulose ether, a cellulose ester, and/or a nitrocellulose.

The at least two radioactive therapeutic agents may be each attached to said core through a covalent bond.

In one embodiment, the particle does not leach radionuclide.

The particles with multiple radionuclides may be used in methods including both radiation treatment and imaging or diagnosing. The use of microparticles with a dual or triple isotope complex allows allows for real-time and post-treatment diagnostic imaging.

The gamma radiation may be assayed to determine the location of the microparticles in the patient.

### EXAMPLES

The following examples are included to demonstrate preferred embodiments of the invention. All of the compositions and methods disclosed can be made and executed without undue experimentation in light of the present disclosure.

While the compositions and methods of this invention have been described in terms of preferred embodiments, it will be apparent to those of skill in the art that variations may be applied to the compositions and/or methods described herein without departing from the concept, spirit and scope of the invention.

More specifically, it will be apparent that certain agents which are both chemically and physiologically related may be substituted for the agents described herein while the same or similar results would be achieved.

All such similar substitutes and modifications apparent to those skilled in the art are deemed to be within the spirit, scope and concept of the invention.

### Example 1

### Preparation of PMMA Microparticulates with Functionalized Surface

PMMA is a hydrophobic polymer but its surface is chemically inert and does not contain functional groups suitable for direct coupling to biologically active substances. PMMA also has low tolerance to organic reagents and solvents. One approach to functionalize PMMA microparticle beads is by hydrolysis of PMMA surface methyl ester groups (Holmberg and Hyden, 1985). This allows for attachment of carrier molecules like dendrimers, containing amino functionalities. Since a dendrimer has multiple functional groups on its surface, the resulting modified microparticulate surface will provide a surface with a high concentration of active sites for further attachment of chelators for labeling. Materials included PMMA microparticulates, 25μ diameter, PAMAM-NH₂ poly(amidoamine) dendrimers, (Sigma), p-NO₂-Bz-DOTA, p-NH₂-Bz-DOTA (Macrocyclic, Tex., USA), 1-ethyl-3-(3-dimethylaminopropyl)carbodiimide EDAC (Fluka) Hydrolysis of PMMA ester groups: To a suspension of PMMA microparticulates in methanol:water (1:1) cooled to 0° C., excess of 10(N) NaOH is added dropwise under stirring. The reaction mixture is stirred at 0° C. for 1.5 h and at 40° C. for 16 h. The microparticulates are washed with 0.1(N) HCl and methanol:water (1:1), centrifuged, decanted and resuspended in PBS (7.4) to obtain PMMA-COOH.

### Example 2

### Activation of PMMA-COOH Spheres and Coupling to PAMAM Dendrimers

PMMA-COOH microparticulates (10 mg) prepared as above are washed 2× in 10 ml PBS. The pellet ia resuspended in 10 ml of PBS and 100 mg of EDAC is added with mixing. The reaction mixture is stirred at room temperature for 15-30 min followed by addition of dendrimer (PAMAM-NH2) (10×) solution in 5 ml PBS. The reaction is allowed to proceed at room temperature for 30min-1h. The microparticulates (PMMA-PAMAM-NH₂) ia washed 2× with PBS and resuspended in PBS. Notes: The reaction/hydrolysis rate of 1-ethyl-3-(3-dimethylaminopropyl)carbodiimide (EDAC) increases with lower pH. Optimum buffer range for reaction is pH 4.5-7.5. EDAC mediated activation of COOH groups and coupling to NH₂—functionality in a single step is often problematic for coupling larger molecules but has been effectively used for smaller molecules like haptens and steroids. EDAC reacts with carboxyls to give an intermediate o-acylisourea. This intermediate reacts with amines to form a peptide bonded conjugate. However the intermediate undergoes hydrolysis in aqueous solutions hence stabilization is usually necessary which may be achieved by adding N-hydroxysuccinimide. To reduce inter dendrimer coupling the concentration of PAMAM should be in large excess. Reference: Holmberg K, Hyden H. Methods of immobilization of proteins to polymethacrylate. Preparative Biochemistry, 15(5): 309-319 (1985).

### Example 3

### Attachment of DOTA Chelator to Dendrimers for Delivery of Yttrium

To a suspension of PMMA-PAMAM-NH₂ microparticulates in PBS, p-NO₂-Bz-DOTA solution in PBS is added dropwise with stirring. The reaction mixture is stirred for 16 h at room temperature. After overnight stirring, the beads are washed 2× with PBS and resuspended in the buffer. Alternatively, direct coupling of DOTA to PMMA-COOH microparticulates can be accomplished using the same procedure as applied to react with p-NH₂-Bz-DOTA (Macrocyclic, Tex., USA)

### Example 4

### Determination of Microparticulate Chelator Concentration

The chelator concentration was determined using constant yttrium-90 (100 μCi) in the presence of variable yttrium-89 to give total yttrium concentrations of 20-1000 μM where yttrium-90 is ≈1 μM. Briefly, yttrium-90 (20 mCi in 100 μL of 50 mM HCl) or yttrium-89 chloride in 50 mM HCl was diluted with 50 μL of 50 mM HCl and 350 μL of 50 mM sodium citrate. In a typical assay, yttrium-89 solution (100-200 μCi, 4 μL), yttrium-89 solution (5 μL), 100 mM histidine buffer containing 10 mM sodium citrate pH 7.4 (25 μL), water (16 μL), and 2 mg/mL microparticulates in 50 mM histidine buffer containing 5 mM sodium citrate at pH 7.4 (50 μL). The yttrium bound to the particulates was determined as described above, and the chelator concentration was determined by extrapolation from a plot of % yttrium bound vs. yttrium concentration. Alternatively, the chelator concentration was determined by adding variable amounts of yttrium-89 to microparticulates followed by yttrium-90.

These titration experiments were performed by adding “cold” yttrium-89 to microparticulates followed by both the addition of the yttrium-90 isotope, and measurement of the yttrium-90 bound to microparticulates. As the amount of yttrium-89 increases, the binding of yttrium-90 decreases due to saturation of the binding sites on the microparticulates which results in inhibition of yttrium-90 binding. The concentration of yttrium-89 at which yttrium-90 no longer binds is equal to the concentration of chelation sites. Alternatively, the titrations were performed by the addition of tracer amounts of yttrium-90 to yttrium-89, and adding this mixture, which contains excess yttrium-89, to microparticulates. Measured concentrations of the DOTA chelator present in solution are in agreement with calculated concentrations. For microparticulates containing 1 and 5 mole percent of the DOTA chelator, the calculated concentrations of 0.11 and 0.55 mM agree closely with the measured concentrations of 0.5 and 0.1 mM of the DOTA chelator.

### Example 5

### Binding of Y-90 to Microparticulates

Naturally occurring yttrium-89 as well as isotopes yttrium-90, and indium-111 are attached to the microparticles via chelation to the DOTA chelating. The labeling efficiency is greater than 98% with a binding capacity for yttrium-90 of approximately 10 mCi per mg of particulate. The effect of pH on yttrium-90 binding efficiency was examined in acetate, MES, and HEPES buffers and is pH independent from pH 5-7. Microparticulates may also be labeled with indium-111, a gamma-emitting isotope commonly used for in-vivo imaging studies. The labeling efficiencies were xx-xx% at loading levels of 50-500 μCi per mg of microparticle. Because of the high metal binding capacity, microparticulates also bind yttrium-90 and indium-111simultaneously. Sequential loading experiments with 0.1 or 1 mCi of each isotope per mg of microparticulate resulted in xx-xx% binding of both isotopes.

### Example 6

### Stability of Y-90 Labeled Microparticulates-Histidine Challenge Studies

Yttrium-90 chloride or indium-111 chloride (10-20 mCi) in 50 mM HCl was diluted with 50 mM citric acid (pH 4) to give a solution that was 50 mCi/mL. To 90 μL of microparticulate solution in 50 mM histidine buffer containing 5 mM citrate at pH 7 was added 10 μL of isotope solution containing 100-200 μCi. The solution was incubated at room temperature for 30 minutes and added to a 100K MWCO spin filter cartridge (Nanosep), which was placed in a table top centrifuge. After spinning at 3000 rpm for 90-120 minutes, the isotope was quantified using a Capintec CRC-15R dose calibrator. The filter portion of the cartridge that contains the microparticulate-isotope complex was removed, and the remaining unbound isotope was quantified. These values were used to calculate the percent metal bound, or the amount of isotope bound per mg of microparticulate.

### Example 7

### Stability of Microparticulate-isotope Conjugates in-vitro

In order to assess the stability of conjugates in serum, the microparticulate 90Y complex containing 5 mole percent chelator was incubated in rabbit serum at 37° C. The solution was incubated at room temperature for 30 minutes and added to a 100K MWCO spin filter cartridge (Nanosep), which was placed in a table top centrifuge. After spinning at 3000 rpm for 90-120 minutes, the isotope was quantified using a Capintec CRC-15R dose calibrator. The filter portion of the cartridge that contains the microparticulate-isotope complex was removed, and the remaining unbound isotope was quantified. These values were used to calculate the percent metal bound, or the amount of isotope bound per mg of microparticulate.

### Example 8

### Specific Y-90 Labeling of the DOTA Chelator

Specific labeling of the DOTA chelator on the vesicles was demonstrated by incubation of the microparticulate 90Y complexes with the weak chelator citrate, and the strong chelator diethylaminetriaminepentaacetic acid (DTPA) at DOTA-lipid concentrations of 0.56-560 μM. The metal complexes are stable in the presence of 500 mM citrate and about 90% of the yttrium is retained in the presence of 1 mM DTPA following a 30-minute incubation of the microparticulate 90Y complex.

The present invention provides a practical way to prepare the agent on-site so that it can be optimized for patient dose requirements and medical facilities and staff schedules. Provides a means to use gamma, beta, and alpha radionuclides alone or in combination on the same particle for radiodetection and/or therapy. Provides a means to accurately measure biodistribution and administered tissue dose. Does not require the product to first be produced in a nuclear reactor off-site, allowing for maximum adaptability for private, university or managed-care medical centers world-wide.

Intra-arterial detection of blood flow distribution and therapy of any organ or intravascularly accessible tumor. Therapy of intravascularly accessible organ or tumor with alpha or beta emitting radionuclides.

### References Cited

1. Kemeny N, Huang Y, Cohen A M, et al. Hepatic Arterial Infusion of Chemotherapy After Resection of Hepatic Metastases From Colorectal Cancer. New England Journal of Medicine 1999; 341:2039-2048.

2. Fong Y, Blumgart L H, Cohen A M. Surgical treatment of colorectal metastases to the liver. C A Cancer J Clin 1995; 45:50-62.

3. El-Serag H B, Mason A C. Rising incidence of hepatocellular carcinoma in the United States. N Engl J Med 1999; 340:745-50.

4. Lawrence T S, Kessler M L, Robertson J M. Conformal high-dose radiation plus intraarterial floxuridine for hepatic cancer. Oncology (Huntingt) 1993; 7:51-7; discussion 57-8, 63.

5. Lawrence T S, Kessler M L, Robertson J M. 3-D conformal radiation therapy in upper gastrointestinal cancer. The University of Michigan experience. Front Radiat Ther Oncol 1996; 29:221-8.

6. Lawrence T S, Dworzanin L M, Walker-Andrews S C, et al. Treatment of cancers involving the liver and porta hepatis with external beam irradiation and intraarterial hepatic fluorodeoxyuridine. Int J Radiat Oncol Biol Phys 1991; 20:555-61.

7. McGinn C J, Ten Haken R K, Ensminger W D, Walker S, Wang S, Lawrence T S. Treatment of intrahepatic cancers with radiation doses based on a normal tissue complication probability model. J Clin Oncol 1998; 16:2246-52.

8. McGinn C J, Lawrence T S. Clinical Results of the Combination of Radiation and Fluoropyrimidines in the Treatment of Intrahepatic Cancer. Semin Radiat Oncol 1997; 7:313-323.

9. Ariel I M. Treatment of inoperable primary pancreatic and liver cancer by the intra-arterial administration of radioactive isotopes (Y90 radiating microspheres). Ann Surg 1965; 162:267-78.

10. Ariel I M, Pack G T. Treatment of inoperable cancer of the liver by intra-arterial radioactive isotopes and chemotherapy. Cancer 1967; 20:793-804.

11. Simon N, Warner R R P, Baron M G, Rudavsky A Z. Intra-arterial Irradiation of Carcinoid Tumors of the Liver. The American Journal of Roentgenology, Radium Therapy and Nuclear Medicine 1968; 102:552-561.

12. Caldarola L, Rosa U, Badellino F. Preparation of 32P Labelled Resin Microspheres For Radiation Treatment of Tumors By Intraarterial Injection. Panminerva Med 1965; 7:102.

13. Blanchard R J, Grotenhuis I, LaFave J W. Treatment of Experimental Tumors: Utilization of Radioactive Microspheres. Archives of Surgery 1964; 89:406.

14. Blanchard R J, LaFave J W, Kim Y S. Treatment of Patients with Advanced Cancer Using Y-90 microspheres. Cancer 1965; 18:375.

15. Kim Y S, LaFave J W, MacLean L D. The Use of Radiating Microspheres in the Treatment of Experimental and Human Malignancy. Surgery 1962; 52:220.

16. Blanchard R J W. Treatment of Liver tumours with yttrium-90 microspheres. The Canadian Journal of Surgery 1983; 26:442-443.

17. Mantravadi R V, Spigos D G, Tan W S, Felix E L. Intraarterial yttrium 90 in the treatment of hepatic malignancy. Radiology 1982; 142:783-6.

18. Ariel I M, Padula G. Treatment of asymptomatic metastatic cancer to the liver from primary colon and rectal cancer by the intraarterial administration of chemotherapy and radioactive isotopes. J Surg Oncol 1982; 20:151-6.

19. Grady E D. Internal radiation therapy of hepatic cancer. Dis Colon Rectum 1979; 22:371-5.

20. Blanchard R J, Morrow I M, Sutherland J B. Treatment of liver tumors with yttrium-90 microspheres alone. Can Assoc Radiol J 1989; 40:206-10.

21. Burton M A, Gray B N, Jones C, Coletti A. Intraoperative dosimetry of 90Y in liver tissue. Int J Rad Appl Instrum B 1989; 16:495-8.

22. Anderson J H, Goldberg J A, Bessent R G, et al. Glass yttrium-90 microspheres for patients with colorectal liver metastases. Radiother Oncol 1992; 25:137-9.

23. Shepherd F A, Rotstein L E, Houle S, Yip T C, Paul K, Sniderman K W. A phase I dose escalation trial of yttrium-90 microspheres in the treatment of primary hepatocellular carcinoma. Cancer 1992; 70:2250-4.

24. Yan Z P, Lin G, Zhao H Y, Dong Y H. An experimental study and clinical pilot trials on yttrium-90 glass microspheres through the hepatic artery for treatment of primary liver cancer. Cancer 1993; 72:3210-5.

25. Andrews J C, Walker S C, Ackermann R J, Cotton L A, Ensminger W D, Shapiro B. Hepatic radioembolization with yttrium-90 containing glass microspheres: preliminary results and clinical follow-up [see comments]. J Nucl Med 1994; 35:1637-44.

26. Lau W Y, Leung W T, Ho S, et al. Treatment of inoperable hepatocellular carcinoma with intrahepatic arterial yttrium-90 microspheres: a phase I and II study. Br J Cancer 1994; 70:994-9.

27. Leung T W, Lau W Y, Ho S K, et al. Radiation pneumonitis after selective internal radiation treatment with intraarterial 90yttrium-microspheres for inoperable hepatic tumors. Int J Radiat Oncol Biol Phys 1995; 33:919-24.

28. Leung W T, Lau W Y, Ho S K, et al. Measuring lung shunting in hepatocellular carcinoma with intrahepatic-arterial technetium-99m macroaggregated albumin. J Nucl Med 1994; 35:70-3.

29. Kennedy A S, Murthy R, Sarfaraz M, et al. Outpatient Hepatic Artery Brachytherapy for Primary and Secondary Hepatic Malignancies. Radiology 2001; 221P:468.

30. Kennedy A S, Murthy R, Van Echo D A. Preliminary Results of Outpatient Hepatic Artery Brachytherapy for Colorectal Hepatic Metastases. European Journal of Cancer 2001; 37:289.

31. Kennedy A S, Murthy R, Kwok Y, al. e. Hepatic Artery Brachytherapy for Unresectable Hepatocellular Carcinoma: An Outpatient Treatment Approach. Proceedings of the 12th International Congress on Anti-Cancer Treatment 2002; 1:198-199.

32. Kennedy A S, Salem R. Comparison of two 90Yttrium microsphere agents for hepatic artery brachytherapy. Proceedings of the 14th International Congress on Anti-Cancer Treatment 2003:156.

33. Kennedy A S, Van Echo D A, Murthy R, al. e. Colorectal (CRC) liver metastases and Hepatocellular carcinoma (HCC) treated with outpatient hepatic artery brachytherapy, TheraSphere: Imaging response and toxicity. Int J Cancer 2002; S13:226-227.

34. Kennedy A S, Van Echo D A, Murthy R, al. e. Hepatic artery brachytherapy for neuroendocrine carcinoma. Regulatory Peptides 2002; 108:32.

35. Murthy R, Kennedy A S, Coldwell D, al. e. Technical aspects of TheraSphere (TS) infusion. J Vasc Interv Radiol 2002; 13:S2.

36. Murthy R, Kennedy A S, Tucker G, al. e. Outpatient trans arterial hepatic ‘low dose rate’ (TAH-LDR) brachytherapy for unresectable hepatocellular carcinoma. Proceedings of American Association for Cancer Research 2002; 43:485.

37. Murthy R, Line B R, Kennedy A S, al. e. Clinical utility of Brehmstralung scan (BRM-Scan) after TheraSphere (TS). J Vasc Interv Radiol 2002; 13:S2.

38. Sarfaraz M, Kennedy A S, Cao Z J, Li A, Yu C. Radiation Dose Distribution in Patients Treated with Y-90 Microspheres for Non-Resectable Hepatic Tumors. International Journal of Radiation Biology and Physics 2001; 51:32-33.

39. Van Echo D A, Kennedy A S, Coldwell D. TheraSphere (TS) at 143 Gy median dose for mixed hepatic cancers; feasibility and toxicities. Amer Soc Clin Oncol 2001; 260a:1038.

40. Coldwell D, Kennedy A S, Van Echo D A, al. e. Feasibility of treatment of hepatic tumors utilizing embolization with yttrium-90 glass microspheres. J Vasc IntervRadiol 2001; 12:S113.

41. Wright A A, Jenkins J J, Lodge M A, Murthy R, Kennedy A S, Line B R. Predictive factors in 90Y microsphere therapy for shunt fraction. J Nuclear Medicine 2002; 43:103P.

42. Mourtzikos K, Lodge M A, Maragh M, et al. Measurement of Hepatopulmonary shunt fraction prior to 90Y microsphere treatment of hepatic malignancy. J Nuclear Medicine 2002; 43:317P.

43. Hisley C, Lodge M A, Kennedy A S, Mourtzikos K, Line B R. Estimation of 90Y TheraSphere Treatment Effectiveness by Statistical Segmentation of Hepatic Tissue Volumes. J Nuclear Medicine 2002; 43:103P.

44. Häfeli U O, Casillas S, Dietz D W, et al. Hepatic tumor radioembolization in a rat model using radioactive rhenium (186RE/188RE) glass microspheres. Int J Radiat Oncol Biol Phys 1999; 44:189-199.

What is claimed is:

1. A microparticle comprising
a core,
at least one linking carrier on said core, wherein said linking carrier comprises a biocompatible polymer, and
at least one radioactive therapeutic agent covalently bonded to said linking carrier;
wherein said microparticle has a diameter in the range of from 5 to 200 microns and said microparticle is non-biodegradable.

2. The particle of claim 1, wherein said microparticle is not water swellable.

3. The particle of claim 1, wherein said radioactive therapeutic agent is a radionuclide or a radiopharmaceutical.

4. The particle of claim 1, wherein said at least one radioactive therapeutic agent is at least one radionuclide selected from the group consisting of an alpha-emitting radionuclide, a beta-emitting radionuclide and a gamma-emitting radionuclide.

5. The particle of claim 1, wherein said at least one radioactive therapeutic agent is an alpha-emitting radionuclide and a beta-emitting radionuclide.

6. The particle of claim 1, wherein said at least one radioactive therapeutic agent is an alpha-emitting radionuclide, a beta-emitting radionuclide and a gamma-emitting radionuclide.

7. The particle of claim 5, wherein said radioactive therapeutic agent is at least one radionuclide selected from the group consisting of iridium, radium, cesium, phosphorus, yttrium, rhenium, actinium, bismuth, astatine, technetium, indium, iodine, and carbon, nitrogen, fluorine, sodium, magnesium, aluminum, silicon, potassium, vanadium, manganese, gallium, niobium, iodine, lead, Y-90, Bi-213, At-211, I-123, I-125, I-131, At-211, Cu-67, Sc-47, Ga-67, Rh-105, Pr-142, Nd-147, Pm-151, Sm-153, Ho-166, Gd-159, Th-161, Eu-152, Er-171, Re-186, Re-188, Tc-99m, In-111, Ga-67, Rh-105, I-123, Nd-147, Pm-151, Sm-153, Gd-159, Th-161, Er-171, Re-186, Re-188, and T1-201.

8. The particle of claim 1, wherein said radioactive therapeutic agent is yttrium-90.

9. The particle of claim 1, wherein said at least one radioactive therapeutic agent comprises a therapeutic radionuclide and an imaging or diagnostic radionuclide both chemically bonded to said linking carrier.

10. The particle of claim 9, wherein said therapeutic radionuclide is a beta-emitting radionuclide and said an imaging or diagnostic radionuclide is a gamma-emitting radionuclide.

11. The particle of claim 10, wherein said therapeutic radionuclide is yttrium-90 and said an imaging or diagnostic radionuclide is selected from the group consisting of indium-111 and Tc-99m.

12. The particle of claim 1, wherein said radioactive therapeutic agent is bonded to said linking carrier through one or more spacer groups.

13. The particle of claim 1, wherein said radioactive therapeutic agent is bound to said linking carrier by a chelator group.

14. The particle of claim 13, wherein said chelator group is at least one selected from the group consisting of cyclohexyldiethylenetriaminepentaacetic acid ligand (CHX-DTPA), diethylenetriaminepentaacetic acid (DTPA), ethylenediaminetetraacetic acid (EDTA), 1,4,7,10-tetraazacyclododecane-N,N′,N,″N′″tetraacetate (DOTA), tetraazacyclotetradecane-N,N″, N″N″-tetraacetic acid (TETA), cyclohexyl 1,2-diamine tetra-acetic acid (CDTA), ethyleneglycol-O,O′-bis(-2-aminoethyl)-N,N,N′,N′-tetra-acetic acid (EGTA), N,N-bis(hydroxybenzyl)-e-thylenediamine-N,N′-diacetic acid (HBED), triethylene tetramine hexa-acetic acid (TTHA), hydroxyethyldiamine triacetic acid (HEDTA), hydroxyethylidene diphosphonate (HEDP), dimercaptosuccinic acid (DMSA), diethylenetriaminetetramethylenephosphonic acid (DTTP) and 1-(p-aminobenzyl)-DTPA, 1,6-diamino hexane-N,N,N′,N′-tetraacetic acid, DPDP, and ethylenebis (oxyethylenenitrilo)-tetraacetic acid.

15. The particle of claim 13, wherein said radioactive therapeutic agent is yttrium-90 and said chelator group is DOTA.

16. The particle of claim 1, wherein said core is non-ceramic and non-radioactively labeled.

17. The particle of claim 1, wherein said core comprises a polymer selected from the group consisting of polyacrylate, ethylene-vinyl acetate polymer, an acyl substituted cellulose acetate, polyurethane, polystyrene, polyvinylchloride, polyvinyl flouride, poly(vinyl imidazole), chlorosulphonate polyolefin, polyethylene oxide, blends thereof, and copolymers thereof, a polyphosphazine, a poly(vinyl alcohol), a polyamide, a polycarbonate, a polyalkylene, a polyacrylamide, a polyalkylene glycol, a polyalkylene oxide, a polyalkylene terephthalate, a polyvinyl ether, a polyvinyl ester, a polyvinyl halide, polyvinylpyrrolidone, a polyglycolide, a polysiloxane, and copolymers thereof, a alkyl cellulose, an hydroxyalkyl cellulose, a cellulose ether, a cellulose ester, and a nitrocellulose.

18. The particle of claim 1, wherein said at least one linking carrier is selected from the group consisting of a linear polymer, a branched polymer, and a dendromer polymer.

19. The particle of claim 18, wherein said at least one linking carrier is a dendrimer.

20. The particle of claim 19, wherein said dendrimer has a disulfide bond in its core.

21. The particle of claim 19, wherein said dendrimer has a final external layer which is capped with a reactive group.

22. The particle of claim 21, wherein said reactive group is an amine or carboxyl group.

23. The particle of claim 21, wherein said reactive group is derivatized with at least one selected from the group consisting of a targeting entity and a therapeutic entity.

24. The particle of claim 19, wherein said dendrimer has a terminal functional group which is accessible to a chelate containing compound which is capable of interacting with the functional groups.

25. The particle of claim 24, wherein said functional group is at least one selected from the group consisting of ester group, ether group, thiol group, carbonyl group, hydroxyl group, amide group, carboxylic group, and imide group.

26. The particle of claim 19, comprising multiple dendrimers, wherein said dendrimers are monodispersed.

27. The particle of claim 18, wherein said linking carriers are linear polymers.

28. The particle of claim 1, wherein said radioactive therapeutic agent is covalently bonded to said linking carrier via a bifunctional linker, carbodiimide condensation, or a disulfide bond formation.

29. The particle of claim 1, wherein said particle does not leach radionuclide.

30. The particle of claim 1, wherein said particle is spheroidal.

31. The particle of claim 1, wherein said particle has a density in the range of from 1 to 4 gm/cm³.

32. The particle of claim 1, wherein said particle has a density in the range of from 1 to 2 gm/cm³.

33. The particle of claim 1, wherein said particle further comprises a second therapeutic agent or a diagnostic agent.

34. The particle of claim 33, wherein said second therapeutic agent or said diagnostic agent is at least one selected from the group consisting of a metal chelate complex, a drug, a prodrug, a radionuclide, a boron addend, a labeling compound, a toxin, a cytokine, a lymphokine, a chemokine, an immunomodulator, a radiosensitizer, an asparaginase, a radioactive halogens, a chemotherapy drug and a contrast agent.

35. A particulate material comprising microparticles having:
a core,
at least one linking carrier on said core, wherein said linking carrier comprises a biocompatible polymer, and
at least one radioactive therapeutic agent covalently bonded to said linking carrier;
wherein said microparticles have a diameter in the range of from 5 to 200 microns and said microparticles are non-biodegradable.

36. The particulate material of claim 35, wherein said microparticles have a diameter in the range of from 8-100 microns.

37. The particulate material of claim 35, wherein said microparticles have a diameter in the range of from 25-50 microns.

38. The particulate material of claim 35, wherein said microparticles have a diameter in the range of from 20-30 microns.

39. The particulate material of claim 35, wherein said microparticles have substantially equivalent particle sizes.

40. The particulate material of claim 35, wherein said microparticles are sufficiently large so as to avoid phagocytosis.

41. A method of radiation therapy of a patient, which comprises administering to the patient microparticles, wherein said microparticles comprise
a core,
at least one linking carrier on said core, wherein said linking carrier comprises a biocompatible polymer, and
at least one radioactive therapeutic agent covalently bonded to said linking carrier;
wherein said microparticle has a diameter in the range of from 5 to 200 microns and said microparticle is non-biodegradable.

42. The method of radiation therapy of a patient of claim 41, wherein said microparticles are administered internally.

43. The method of radiation therapy of a patient of claim 41, wherein the administration is direct to a lesion or through a vascular route.

44. The method of radiation therapy of a patient of claim 41, wherein said radiation therapy treats cancer or a tumor.

45. The method of radiation therapy of a patient of claim 44, wherein said cancer is primary or secondary cancer of the liver.

46. The method of radiation therapy of a patient of claim 41, wherein said radiation therapy treats a highly vascularized tumor or a tumor which has a single dominant arterial vascular supply.

47. The method of radiation therapy of a patient of claim 46, wherein said microparticles are injected into an artery supplying a tumor.

48. The method of radiation therapy of a patient of claim 41, wherein said radiation therapy treats hepatic cancer, rheumatoid arthritis, a solid cancer, liver cancer, brain cancer, breast cancer and/or ovary cancer.

49. The method of radiation therapy of a patient of claim 41, wherein said radiation therapy treats renal cell carcinoma, hepatoma, sarcomas, cancer of the head or neck, and/or a central nervous system tumor.

50. The method of radiation therapy of a patient of claim 41, wherein said at least one radioactive therapeutic agent is at least one radionuclide selected from the group consisting of an alpha-emitting radionuclide, a beta-emitting radionuclide and a gamma-emitting radionuclide.

51. The method of radiation therapy of a patient of claim 50, wherein said at least one radioactive therapeutic agent is an alpha-emitting radionuclide and a beta-emitting radionuclide.

52. The method of radiation therapy of a patient of claim 51, wherein said at least one radioactive therapeutic agent is an alpha-emitting radionuclide, a beta-emitting radionuclide and a gamma-emitting radionuclide.

53. The method of radiation therapy of a patient of claim 41, comprising radiation treatment and imaging or diagnosing.

54. The method of radiation therapy of a patient of claim 53, wherein said imaging or diagnosing is during the life of the radiation.

55. The method of radiation therapy of a patient of claim 53, wherein said imaging or diagnosing is post life of the radiation.

56. The method of radiation therapy of a patient of claim 51, further comprising assaying the gamma radiation to determine the location of the microparticles in the patient.

57. The method of radiation therapy of a patient of claim 41, wherein said particles are immobilized at a site of administration.

58. The method of radiation therapy of a patient of claim 41, wherein said particles do not release a significant amount of radiation emmitting radioisotope into the circulation system upon administration.

59. The method of radiation therapy of a patient of claim 41, wherein said particles have a diameter of from 15 to 35 microns.

60. A kit for preparing a microparticle treatment dose for a patient in need thereof, wherein said treatment dose comprises the particulate material of claim 35, wherein said kit comprises
particle cores, which do not comprise radionuclide,
linkers for attaching at least one radionuclide to said particle cores, and
instructions or a means for obtaining instructions for preparing said microparticle treatment dose.

61. The kit for preparing a microparticle treatment dose of claim 60, wherein said kit contains a radionuclide.

62. The kit for preparing a microparticle treatment dose of claim 60, wherein a radionuclide is provided separately from said kit.

63. The kit for preparing a microparticle treatment dose of claim 60, further comprising at least one component selected from the group consisting of an inert pharmaceutically acceptable carrier, a formulating agent, an adjuvant, an active agent, water, saline, a transfer ligand, a reducing agent, a lyophilization aid, a stabilization aid, a solubilization aid, a bacteriostat, a buffer, an X-ray contrast agent, an ultrasound contrast agent, and a metallopharmaceutical.

64. The kit for preparing a microparticle treatment dose of claim 60, further comprising at least one component selected from the group consisting of a syringe, shielding, and imaging equipment.

65. The kit for preparing a microparticle treatment dose of claim 60, wherein said kit comprises multiple types of cores and multiple types of linkers.

66. A method of using the kit of claim 60 to prepare a microparticle treatment dose for a patient in need thereof,
determining the type and dosimety of microparticle treatment needed from a prescription for said patient and preparing said microparticle treatment dose from said instructions or said means for obtaining instructions.

67. A method of using the kit of claim 60 to prepare a microparticle treatment dose for a patient in need thereof,
determining the type and dosimety of microparticle treatment needed from a prescription for said patient,
selecting a type of core from the cores included in said kit,
selecting a type of linker from the linkers included in said kit,
selecting a radionuclide and
preparing said microparticle treatment dose from said instructions or said means for obtaining instructions.

68. The method of claim 60, wherein said microparticle treatment dose is made said kit at a location of administration or at a site proximate to the location of administration.

69. The method of claim 60, wherein said location or said site is a local radiopharmacy, laboratory, hospital or physician's office.

70. The method of claim 60, wherein said microparticle treatment dose are made said kit at a location of administration or at a site proximate to the location of administration.

71. The method of claim 60, wherein said location or said site is a local radiopharmacy, laboratory, hospital or physician's office.

72. A microparticle comprising
a core, and
at least two radioactive therapeutic agents attached to said core.

73. The particle of claim 72, wherein said at least two radioactive therapeutic agents are independently selected from the group consisting of a therapeutic radionuclide and an imaging or diagnostic radionuclide.

74. The particle of claim 72, wherein at least one of said at least two radioactive therapeutic agents is a beta-emitting radionuclide and at least one of said at least two radioactive therapeutic agents is a gamma-emitting radionuclide.

75. The particle of claim 74, wherein said beta-emitting radionuclide is a therapeutic radionuclide and said gamma-emitting radionuclide is an imaging or diagnostic radionuclide.

76. The particle of claim 75, wherein said therapeutic radionuclide is yttrium-90 and said an imaging or diagnostic radionuclide is selected from the group consisting of indium-111 and Tc-99m.

77. The particle of claim 72, wherein said core is non-ceramic and non-radioactively labeled.

78. The particle of claim 72, wherein said core comprises a polymer selected from the group consisting of polyacrylate, ethylene-vinyl acetate polymer, an acyl substituted cellulose acetate, polyurethane, polystyrene, polyvinylchloride, polyvinyl flouride, poly(vinyl imidazole), chlorosulphonate polyolefin, polyethylene oxide, blends thereof, and copolymers thereof, a polyphosphazine, a poly(vinyl alcohol), a polyamide, a polycarbonate, a polyalkylene, a polyacrylamide, a polyalkylene glycol, a polyalkylene oxide, a polyalkylene terephthalate, a polyvinyl ether, a polyvinyl ester, a polyvinyl halide, polyvinylpyrrolidone, a polyglycolide, a polysiloxane, and copolymers thereof, a alkyl cellulose, an hydroxyalkyl cellulose, a cellulose ether, a cellulose ester, and a nitrocellulose.

79. The particle of claim 72, wherein said at least two radioactive therapeutic agents are each attached to said core through a covalent bond.

80. The particle of claim 72, wherein said particle does not leach radionuclide.

81. The particle of claim 72, wherein said at least two radioactive therapeutic agents are independently selected from the group consisting of an alpha-emitting radionuclide, a beta-emitting radionuclide and a gamma-emitting radionuclide.

82. A method of radiation therapy of a patient, which comprises administering to the patient microparticles, wherein said microparticles comprise the microparticle as claimed in claim 72.

83. The method of radiation therapy of a patient of claim 82, comprising radiation treatment and imaging or diagnosing.

84. The method of radiation therapy of a patient of claim 82, further comprising assaying the gamma radiation to determine the location of the microparticles in the patient.

85. The particle of claim 1, wherein said microparticle has a diameter in the range of from 8 to 100.

86. The particle of claim 1, wherein said microparticle has a diameter in the range of from 20 to 30.
